(19) United States
(12) Patent Application Publication
(10) Pub. No.: US 2026/0086080 A1
(43) Pub. Date: Mar. 26, 2026

(54) PRODUCTION AND APPLICATIONS OF OVARIAN AND UTERINE ORGANOIDS

(71) Applicant: Gameto, Inc., New York, NY (US)

(72) Inventors: Christian KRAMME, New York, NY (US); Dina RADENKOVIC, New York, NY (US)

(21) Appl. No.: 19/109,690

(22) PCT Filed: Sep. 8, 2023
(86) PCT No.: PCT/US2023/032312

§ 371 Date: Mar. 7, 2025

Related U.S. Application Data
(63) Continuation of application No. 17/941,423, filed on Sep. 9, 2022, now abandoned.

(51) Int. Cl. G01N 33/50; C12N 5/071

(52) U.S. Cl. CPC G01N 33/5044; C12N 5/0682; C12N 2506/45; G01N 2800/364

(57) ABSTRACT

Featured are compositions, methods, and apparatuses for biosynthetic organs, or organoids, that replicate tissues of the human female reproductive system (e.g., ovarian or uterine tissues). In particular, the disclosure features methods of culturing or 2024/054644 producing organoids or uteroids from pluripotent stem or progenitor cells and using the resulting organoids or uteroids to screen for safe and effective pharmaceutical interventions for diseases of interest. Such methods and compositions are particularly useful for the treatment of patients undergoing hormone replacement therapy (HRT) and/or assisted reproduction technology (ART) procedures, such as in vitro fertilization and in vitro oocyte maturation.

WO

### TECHNICAL FIELD

This disclosure relates to the field of ovarian and uterine synthetic biology, particularly in the context of developing organoid models of female reproductive tissue useful for screening candidate pharmaceutical interventions for safety and efficacy in treating various diseases and conditions.

### BACKGROUND

One in ten women struggle with infertility, often requiring assisted reproductive technology (ART), such as in vitro fertilization (IVF) or forms of hormone replacement therapy (HRT), in order to augment their likelihood of successful conception and maintaining pregnancy. A wide variety of mechanisms underlie the ovarian and uterine conditions that hinder fertility, and challenges remain in studying these mechanisms. These challenges include limitations on the availability of suitable animal models, confounding variables from existing in vivo data, and overly simplistic representations of in vitro cell culture models that utilize single-cell populations to replicate a complex, multicellular environment of the human female reproductive system. Accordingly, there remains a need for effective ex vivo models for evaluating the safety and efficacy of potential therapeutic interventions, as well as a need for new models of the female reproductive system for elucidating mechanisms of humane disease.

### SUMMARY OF THE INVENTION

In a first aspect, the disclosure features an organoid containing a population of cells of a human female reproductive organ. In some embodiments, the organoid further includes an extracellular matrix (ECM) substrate. The cells may be obtained, e.g., by differentiation, ex vivo, of one or more pluripotent stem or progenitor cells, and the population of cells may include: (a) a plurality of ovarian granulosa cells, ovarian stroma cells, ovarian lutein cells, and/or ovarian theca cells; and/or (b) a plurality of uterine endometrial cells, uterine myometrial cells, and/or uterine perimetrial cells.

In some embodiments, the organ is an ovary. In some embodiments, the population of cells includes a plurality of ovarian granulosa cells, ovarian stroma cells, ovarian lutein cells, and/or ovarian theca cells.

In some embodiments, the population of cells includes a plurality of ovarian granulosa cells. In some embodiments, the ovarian granulosa cells express one or more, or all, of proteins CD82, follicle-stimulating hormone receptor (FSHR), FOXL2, NR5A1, GATA4, RUNX1, and RUNX2. In some embodiments, the ovarian granulosa cells secrete an estrogen, such as estradiol.

In some embodiments, the population of cells includes from about 1×10⁵ to about 1×10⁸ ovarian granulosa cells. In some embodiments, the population of cells includes from about 1×10⁵ to about 2×10⁵ ovarian granulosa cells, from about 2×10⁵ to about 3×10⁵ ovarian granulosa cells, from about 3×10⁵ to about 4×10⁵ ovarian granulosa cells, from about 4×10⁵ to about 5×10⁵ ovarian granulosa cells, from about 5×10⁵ to about 6×10⁵ ovarian granulosa cells, from about 6×10⁵ to about 7×10⁵ ovarian granulosa cells, from about 7×10⁵ to about 8×10⁵ ovarian granulosa cells, from about 8×10⁵ to about 9×10⁵ ovarian granulosa cells, or from about 9×10⁵ to about 1×10⁶ ovarian granulosa cells.

In some embodiments, the population of cells includes from about 1×10⁶ to about 2×10⁶ ovarian granulosa cells, from about 2×10⁶ to about 3×10⁶ ovarian granulosa cells, from about 3×10⁶ to about 4×10⁶ ovarian granulosa cells, from about 4×10⁶ to about 5×10⁶ ovarian granulosa cells, from about 5×10⁶ to about 6×10⁶ ovarian granulosa cells, from about 6×10⁶ to about 7×10⁶ ovarian granulosa cells, from about 7×10⁶ to about 8×10⁶ ovarian granulosa cells, from about 8×10⁶ to about 9×10⁶ ovarian granulosa cells, or from about 9×10⁶ to about 1×10⁷ ovarian granulosa cells.

In some embodiments, the population of cells includes from about 1×10⁷ to about 2×10⁷ ovarian granulosa cells, from about 2×10⁷ to about 3×10⁷ ovarian granulosa cells, from about 3×10⁷ to about 4×10⁷ ovarian granulosa cells, from about 4×10⁷ to about 5×10⁷ ovarian granulosa cells, from about 5×10⁷ to about 6×10⁷ ovarian granulosa cells, from about 6×10⁷ to about 7×10⁷ ovarian granulosa cells, from about 7×10⁷ to about 8×10⁷ ovarian granulosa cells, from about 8×10⁷ to about 9×10⁷ ovarian granulosa cells, or from about 9×10⁷ to about 1×10⁸ ovarian granulosa cells.

In some embodiments, the population of cells includes about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian granulosa cells.

In some embodiments, the population of cells includes a plurality of ovarian stroma cells. In some embodiments, the ovarian stroma cells express NR2F2.

In some embodiments, the population of cells includes from about 1×10⁵ to about 1×10⁸ ovarian stroma cells. In some embodiments, the population of cells includes from about 1×10⁵ to about 2×10⁵ ovarian stroma cells, from about 2×10⁵ to about 3×10⁵ ovarian stroma cells, from about 3×10⁵ to about 4×10⁵ ovarian stroma cells, from about 4×10⁵ to about 5×10⁵ ovarian stroma cells, from about 5×10⁵ to about 6×10⁵ ovarian stroma cells, from about 6×10⁵ to about 7×10⁵ ovarian stroma cells, from about 7×10⁵ to about 8×10⁵ ovarian stroma cells, from about 8×10⁵ to about 9×10⁵ ovarian stroma cells, or from about 9×10⁵ to about 1×10⁶ ovarian stroma cells.

In some embodiments, the population of cells includes from about 1×10⁶ to about 2×10⁶ ovarian stroma cells, from about 2×10⁶ to about 3×10⁶ ovarian stroma cells, from about 3×10⁶ to about 4×10⁶ ovarian stroma cells, from about 4×10⁶ to about 5×10⁶ ovarian stroma cells, from about 5×10⁶ to about 6×10⁶ ovarian stroma cells, from about 6×10⁶ to about 7×10⁶ ovarian stroma cells, from about 7×10⁶ to about 8×10⁶ ovarian stroma cells, from about 8×10⁶ to about 9×10⁶ ovarian stroma cells, or from about 9×10⁶ to about 1×10⁷ ovarian stroma cells.

In some embodiments, the population of cells includes from about 1×10⁷ to about 2×10⁷ ovarian stroma cells, from about 2×10⁷ to about 3×10⁷ ovarian stroma cells, from about 3×10⁷ to about 4×10⁷ ovarian stroma cells, from about 4×10⁷ to about 5×10⁷ ovarian stroma cells, from about 5×10⁷ to about 6×10⁷ ovarian stroma cells, from about 6×10⁷ to about 7×10⁷ ovarian stroma cells, from about 7×10⁷ to about 8×10⁷ ovarian stroma cells, from about 8×10⁷ to about 9×10⁷ ovarian stroma cells, or from about 9×10⁷ to about 1×10⁸ ovarian stroma cells.

In some embodiments, the population of cells includes about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian stroma cells.

In some embodiments, the population of cells includes a plurality of ovarian lutein cells. In some embodiments, the ovarian lutein cells express one or more, or all, of proteins KRT19, CYP19A1, STAR, CYP17A1, and PGR. In some embodiments, the ovarian lutein cells secrete a progestogen, such as progesterone.

In some embodiments, the population of cells includes from about 1×10⁵ to about 1×10⁸ ovarian lutein cells. In some embodiments, the population of cells includes from about 1×10⁵ to about 2×10⁵ ovarian lutein cells, from about 2×10⁵ to about 3×10⁵ ovarian lutein cells, from about 3×10⁵ to about 4×10⁵ ovarian lutein cells, from about 4×10⁵ to about 5×10⁵ ovarian lutein cells, from about 5×10⁵ to about 6×10⁵ ovarian lutein cells, from about 6×10⁵ to about 7×10⁵ ovarian lutein cells, from about 7×10⁵ to about 8×10⁵ ovarian lutein cells, from about 8×10⁵ to about 9×10⁵ ovarian lutein cells, or from about 9×10⁵ to about 1×10⁶ ovarian lutein cells.

In some embodiments, the population of cells includes from about 1×10⁶ to about 2×10⁶ ovarian lutein cells, from about 2×10⁶ to about 3×10⁶ ovarian lutein cells, from about 3×10⁶ to about 4×10⁶ ovarian lutein cells, from about 4×10⁶ to about 5×10⁶ ovarian lutein cells, from about 5×10⁶ to about 6×10⁶ ovarian lutein cells, from about 6×10⁶ to about 7×10⁶ ovarian lutein cells, from about 7×10⁶ to about 8×10⁶ ovarian lutein cells, from about 8×10⁶ to about 9×10⁶ ovarian lutein cells, or from about 9×10⁶ to about 1×10⁷ ovarian lutein cells.

In some embodiments, the population of cells includes from about 1×10⁷ to about 2×10⁷ ovarian lutein cells, from about 2×10⁷ to about 3×10⁷ ovarian lutein cells, from about 3×10⁷ to about 4×10⁷ ovarian lutein cells, from about 4×10⁷ to about 5×10⁷ ovarian lutein cells, from about 5×10⁷ to about 6×10⁷ ovarian lutein cells, from about 6×10⁷ to about 7×10⁷ ovarian lutein cells, from about 7×10⁷ to about 8×10⁷ ovarian lutein cells, from about 8×10⁷ to about 9×10⁷ ovarian lutein cells, or from about 9×10⁷ to about 1×10⁸ ovarian lutein cells.

In some embodiments, the population of cells includes about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian lutein cells.

In some embodiments, the population of cells includes a plurality of ovarian theca cells. In some embodiments, the ovarian theca cells express one or both of proteins NR2F2 and GATA4. In some embodiments, the ovarian theca cells secrete an androgen, such as androstenedione.

In some embodiments, the population of cells includes from about 1×10⁵ to about 1×10⁸ ovarian theca cells. In some embodiments, the population of cells includes from about 1×10⁵ to about 2×10⁵ ovarian theca cells, from about 2×10⁵ to about 3×10⁵ ovarian theca cells, from about 3×10⁵ to about 4×10⁵ ovarian theca cells, from about 4×10⁵ to about 5×10⁵ ovarian theca cells, from about 5×10⁵ to about 6×10⁵ ovarian theca cells, from about 6×10⁵ to about 7×10⁵ ovarian theca cells, from about 7×10⁵ to about 8×10⁵ ovarian theca cells, from about 8×10⁵ to about 9×10⁵ ovarian theca cells, or from about 9×10⁵ to about 1×10⁶ ovarian theca cells.

In some embodiments, the population of cells includes from about 1×10⁶ to about 2×10⁶ ovarian theca cells, from about 2×10⁶ to about 3×10⁶ ovarian theca cells, from about 3×10⁶ to about 4×10⁶ ovarian theca cells, from about 4×10⁶ to about 5×10⁶ ovarian theca cells, from about 5×10⁶ to about 6×10⁶ ovarian theca cells, from about 6×10⁶ to about 7×10⁶ ovarian theca cells, from about 7×10⁶ to about 8×10⁶ ovarian theca cells, from about 8×10⁶ to about 9×10⁶ ovarian theca cells, or from about 9×10⁶ to about 1×10⁷ ovarian theca cells.

In some embodiments, the population of cells includes from about 1×10⁷ to about 2×10⁷ ovarian theca cells, from about 2×10⁷ to about 3×10⁷ ovarian theca cells, from about 3×10⁷ to about 4×10⁷ ovarian theca cells, from about 4×10⁷ to about 5×10⁷ ovarian theca cells, from about 5×10⁷ to about 6×10⁷ ovarian theca cells, from about 6×10⁷ to about 7×10⁷ ovarian theca cells, from about 7×10⁷ to about 8×10⁷ ovarian theca cells, from about 8×10⁷ to about 9×10⁷ ovarian theca cells, or from about 9×10⁷ to about 1×10⁸ ovarian theca cells.

In some embodiments, the population of cells includes about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian theca cells.

In some embodiments, the germ cells include one or more human primordial germ cell-like cells (hPGCLCs), oogonia, and/or oocytes. In some embodiments, the germ cells comprise one or more hPGCLCs, oogonia, and oocytes.

In some embodiments, the germ cells include one or more hPGCLCs. In some embodiments the hPGCLCs express one or more, or all, of proteins NANOS3, CD38, ITGA6, EpCAM, BLIMP1, TFAP2C and SOX17.

In some embodiments, the population of cells includes from about 1×10⁵ to about 1×10⁸ hPGCLCs.

In some embodiments, the population of cells includes from about 1×10⁵ to about 2×10⁵ hPGCLCs, from about 2×10⁵ to about 3×10⁵ hPGCLCs, from about 3×10⁵ to about 4×10⁵ hPGCLCs, from about 4×10⁵ to about 5×10⁵ hPGCLCs, from about 5×10⁵ to about 6×10⁵ hPGCLCs, from about 6×10⁵ to about 7×10⁵ hPGCLCs, from about 7×10⁵ to about 8×10⁵ hPGCLCs, from about 8×10⁵ to about 9×10⁵ hPGCLCs, or from about 9×10⁵ to about 1×10⁶ hPGCLCs.

In some embodiments, the population of cells includes from about 1×10⁶ to about 2×10⁶ hPGCLCs, from about 2×10⁶ to about 3×10⁶ hPGCLCs, from about 3×10⁶ to about 4×10⁶ hPGCLCs, from about 4×10⁶ to about 5×10⁶ hPGCLCs, from about 5×10⁶ to about 6×10⁶ hPGCLCs, from about 6×10⁶ to about 7×10⁶ hPGCLCs, from about 7×10⁶ to about 8×10⁶ hPGCLCs, from about 8×10⁶ to about 9×10⁶ hPGCLCs, or from about 9×10⁶ to about 1×10⁷ hPGCLCs.

In some embodiments, the population of cells includes from about 1×10⁷ to about 2×10⁷ hPGCLCs, from about 2×10⁷ to about 3×10⁷ hPGCLCs, from about 3×10⁷ to about 4×10⁷ hPGCLCs, from about 4×10⁷ to about 5×10⁷ hPGCLCs, from about 5×10⁷ to about 6×10⁷ hPGCLCs, from about 6×10⁷ to about 7×10⁷ hPGCLCs, from about 7×10⁷ to about 8×10⁷ hPGCLCs, from about 8×10⁷ to about 9×10⁷ hPGCLCs, or from about 9×10⁷ to about 1×10⁸ hPGCLCs.

In some embodiments, the population of cells includes about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ hPGCLCs.

In some embodiments, the germ cells include one or more oogonia. In some embodiments, the oogonia express one or more, or all, of proteins DDX4, DAZL, and STRA8.

In some embodiments, the population of cells includes from about 1×10⁵ to about 1×10⁸ oogonia.

In some embodiments, the population of cells includes from about 1×10⁵ to about 2×10⁵ oogonia, from about 2×10⁵ to about 3×10⁵ oogonia, from about 3×10⁵ to about 4×10⁵ oogonia, from about 4×10⁵ to about 5×10⁵ oogonia, from about 5×10⁵ to about 6×10⁵ oogonia, from about 6×10⁵ to about 7×10⁵ oogonia, from about 7×10⁵ to about 8×10⁵ oogonia, from about 8×10⁵ to about 9×10⁵ oogonia, or from about 9×10⁵ to about 1×10⁶ oogonia.

In some embodiments, the population of cells includes from about 1×10⁶ to about 2×10⁶ oogonia, from about 2×10⁶ to about 3×10⁶ oogonia, from about 3×10⁶ to about 4×10⁶ oogonia, from about 4×10⁶ to about 5×10⁶ oogonia, from about 5×10⁶ to about 6×10⁶ oogonia, from about 6×10⁶ to about 7×10⁶ oogonia, from about 7×10⁶ to about 8×10⁶ oogonia, from about 8×10⁶ to about 9×10⁶ oogonia, or from about 9×10⁶ to about 1×10⁷ oogonia.

In some embodiments, the population of cells includes from about 1×10⁷ to about 2×10⁷ oogonia, from about 2×10⁷ to about 3×10⁷ oogonia, from about 3×10⁷ to about 4×10⁷ oogonia, from about 4×10⁷ to about 5×10⁷ oogonia, from about 5×10⁷ to about 6×10⁷ oogonia, from about 6×10⁷ to about 7×10⁷ oogonia, from about 7×10⁷ to about 8×10⁷ oogonia, from about 8×10⁷ to about 9×10⁷ oogonia, or from about 9×10⁷ to about 1×10⁸ oogonia.

In some embodiments, the population of cells includes about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ oogonia.

In some embodiments, the germ cells include one or more oocytes. In some embodiments, the oocytes express one or more, or all, of proteins SYCP1, ZP1, ZP2, REC8, LHX8, and SOHLH1.

In some embodiments, the population of cells includes from about 1×10⁵ to about 1×10⁸ oocytes.

In some embodiments, the population of cells includes from about 1×10⁵ to about 2×10⁵ oocytes, from about 2×10⁵ to about 3×10⁵ oocytes, from about 3×10⁵ to about 4×10⁵ oocytes, from about 4×10⁵ to about 5×10⁵ oocytes, from about 5×10⁵ to about 6×10⁵ oocytes, from about 6×10⁵ to about 7×10⁵ oocytes, from about 7×10⁵ to about 8×10⁵ oocytes, from about 8×10⁵ to about 9×10⁵ oocytes, or from about 9×10⁵ to about 1×10⁶ oocytes.

In some embodiments, the population of cells includes from about 1×10⁶ to about 2×10⁶ oocytes, from about 2×10⁶ to about 3×10⁶ oocytes, from about 3×10⁶ to about 4×10⁶ oocytes, from about 4×10⁶ to about 5×10⁶ oocytes, from about 5×10⁶ to about 6×10⁶ oocytes, from about 6×10⁶ to about 7×10⁶ oocytes, from about 7×10⁶ to about 8×10⁶ oocytes, from about 8×10⁶ to about 9×10⁶ oocytes, or from about 9×10⁶ to about 1×10⁷ oocytes.

In some embodiments, the population of cells includes from about 1×10⁷ to about 2×10⁷ oocytes, from about 2×10⁷ to about 3×10⁷ oocytes, from about 3×10⁷ to about 4×10⁷ oocytes, from about 4×10⁷ to about 5×10⁷ oocytes, from about 5×10⁷ to about 6×10⁷ oocytes, from about 6×10⁷ to about 7×10⁷ oocytes, from about 7×10⁷ to about 8×10⁷ oocytes, from about 8×10⁷ to about 9×10⁷ oocytes, or from about 9×10⁷ to about 1×10⁸ oocytes.

In some embodiments, the pluripotent stem or progenitor cells are induced pluripotent stem cells (iPSCs), such as human iPSCs (hiPSCs).

In some embodiments, the organ is an ovary, the population of cells includes a plurality of ovarian granulosa cells, ovarian stroma cells, ovarian lutein cells, and/or ovarian theca cells, and the population of cells is obtained by modifying the iPSCs (e.g., hiPSCs) to express one or more, or all five, of FOXL2, NR5A1, GATA4, RUNX1, and RUNX2.

In some embodiments, the organoid further includes an ECM substrate. In some embodiments, the ECM substrate includes one or more of collagen, an epidermal growth factor (EGF), an elastin, a fibronectin, a vitronectin, a laminin, a cell adhesion protein, or a plant-derived protein or protein polymer. In some embodiments, the collagen is fibrillar collagen. In some embodiments, the collagen is collagen type I, type II, type III, type V, type XI. In some embodiments, the elastin is tropoelastin or mature elastin. In some embodiments, the plant-derived protein polymer is alginate.

In some embodiments, the organ is a uterus and the population of cells includes a plurality of uterine endometrial cells, uterine myometrial cells, and/or uterine perimetrial cells. In some embodiments, the population of cells includes a plurality of uterine endometrial cells, uterine myometrial cells, and uterine perimetrial cells.

In another aspect, the disclosure features a method of producing an organoid containing a population of human ovarian cells, the method including: (a) introducing, into one or more pluripotent stem or progenitor cells, one or more nucleic acid molecules that collectively encode FOXL2, NR5A1, GATA4, RUNX1, and/or RUNX2, thereby differentiating the one or more pluripotent stem or progenitor cells into a population of ovarian granulosa cells, ovarian stroma cells, ovarian lutein cells, and/or ovarian theca cells; and (b) contacting the cells resulting from (a) with an ECM substrate, optionally wherein the ECM substrate includes one or more of collagen, an EGF, an elastin, a fibronectin, a vitronectin, a laminin, a cell adhesion protein, or a plant-derived protein or protein polymer.

In some embodiments, the one or more nucleic acid molecules are introduced into the one or more pluripotent stem or progenitor cells by way of electroporation. In some embodiments, the one or more nucleic acid molecules collectively encode at least two of FOXL2, NR5A1, GATA4, RUNX1, and RUNX2. In some embodiments, the one or more nucleic acid molecules collectively encode at least three of FOXL2, NR5A1, GATA4, RUNX1, and RUNX2. In some embodiments, the one or more nucleic acid molecules collectively encode at least four of FOXL2, NR5A1, GATA4, RUNX1, and RUNX2. In some embodiments, the one or more nucleic acid molecules collectively encode all five of FOXL2, NR5A1, GATA4, RUNX1, and RUNX2.

In some embodiments, the one or more nucleic acid molecules collectively encode NANOS3, CD38, ITGA6, EpCAM, BLIMP1, TFAP2C, SOX17, or a combination thereof, thereby differentiating the one or more pluripotent stem or progenitor cells into a population of germ cells. In some embodiments, the germ cells comprise one or more hPGCLCs, oogonia, oocytes, or a combination thereof.

In some embodiments, the one or more pluripotent stem or progenitor cells include from about 1×10⁵ to about 1×10⁸ pluripotent stem or progenitor cells. In some embodiments, the one or more pluripotent stem or progenitor cells include from about 1×10⁵ to about 2×10⁵ pluripotent stem or progenitor cells, from about 2×10⁵ to about 3×10⁵ pluripotent stem or progenitor cells, from about 3×10⁵ to about 4×10⁵ pluripotent stem or progenitor cells, from about 4×10⁵ to about 5×10⁵ pluripotent stem or progenitor cells, from about 5×10⁵ to about 6×10⁵ pluripotent stem or progenitor cells, from about 6×10⁵ to about 7×10⁵ pluripotent stem or progenitor cells, from about 7×10⁵ to about 8×10⁵ pluripotent stem or progenitor cells, from about 8×10⁵ to about 9×10⁵ pluripotent stem or progenitor cells, or from about 9×10⁵ to about 1×10⁶ pluripotent stem or progenitor cells.

In some embodiments, the one or more pluripotent stem or progenitor cells include from about 1×10⁶ to about 2×10⁶ pluripotent stem or progenitor cells, from about 2×10⁶ to about 3×10⁶ pluripotent stem or progenitor cells, from about 3×10⁶ to about 4×10⁶ pluripotent stem or progenitor cells, from about 4×10⁶ to about 5×10⁶ pluripotent stem or progenitor cells, from about 5×10⁶ to about 6×10⁶ pluripotent stem or progenitor cells, from about 6×10⁶ to about 7×10⁶ pluripotent stem or progenitor cells, from about 7×10⁶ to about 8×10⁶ pluripotent stem or progenitor cells, from about 8×10⁶ to about 9×10⁶ pluripotent stem or progenitor cells, or from about 9×10⁶ to about 1×10⁷ pluripotent stem or progenitor cells.

In some embodiments, the one or more pluripotent stem or progenitor cells include from about 1×10⁷ to about 2×10⁷ pluripotent stem or progenitor cells, from about 2×10⁷ to about 3×10⁷ pluripotent stem or progenitor cells, from about 3×10⁷ to about 4×10⁷ pluripotent stem or progenitor cells, from about 4×10⁷ to about 5×10⁷ pluripotent stem or progenitor cells, from about 5×10⁷ to about 6×10⁷ pluripotent stem or progenitor cells, from about 6×10⁷ to about 7×10⁷ pluripotent stem or progenitor cells, from about 7×10⁷ to about 8×10⁷ pluripotent stem or progenitor cells, from about 8×10⁷ to about 9×10⁷ pluripotent stem or progenitor cells, or from about 9×10⁷ to about 1×10⁸ pluripotent stem or progenitor cells.

In some embodiments, the one or more pluripotent stem or progenitor cells include about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ pluripotent stem or progenitor cells.

In some embodiments, the pluripotent stem or progenitor cells are iPSCs (e.g., hiPSCs).

In some embodiments, the collagen of the ECM substrate is fibrillar collagen. In some embodiments, the collagen is collagen type I, type II, type III, type V, type XI. In some embodiments, the elastin is tropoelastin or mature elastin. In some embodiments, the plant-derived protein polymer is alginate.

In a further aspect, the disclosure features a method of determining whether a candidate pharmaceutical intervention is efficacious in treating a disease or condition of the human female reproductive system, the method including: (a) contacting the candidate pharmaceutical intervention with the organoid of any one of the foregoing aspects or embodiments of the disclosure; (b) determining that the organoid exhibits (i) an increase (e.g., an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more) in one or more metrics of ovarian and/or uterine function relative to a measurement of the one or more metrics of ovarian and/or uterine function obtained prior to the contacting, and/or (ii) a decrease (e.g., a decrease of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more) in one or more metrics of severity of the disease or condition relative to a measurement of the one or more metrics of severity of the disease or condition obtained prior to the contacting; and, optionally, (c) releasing the candidate pharmaceutical intervention for treatment of the disease or condition in a subject in need thereof.

In some embodiments, the method further includes: (d) administering a therapeutically effective amount of the candidate pharmaceutical intervention to the subject.

In another aspect, the disclosure features a method of treating or preventing a disease or condition of the human female reproductive system in a subject in need thereof, the method including: (a) contacting the candidate pharmaceutical intervention with the organoid of any one of the foregoing aspects or embodiments of the disclosure; (b) determining that the organoid exhibits (i) an increase (e.g., an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more) in one or more metrics of ovarian and/or uterine function relative to a measurement of the one or more metrics of ovarian and/or uterine function obtained prior to the contacting, and/or (ii) a decrease (e.g., a decrease of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more) in one or more metrics of severity of the disease or condition relative to a measurement of the one or more metrics of severity of the disease or condition obtained prior to the contacting; and (c) administering a therapeutically effective amount of the candidate pharmaceutical intervention to the subject.

In another aspect, the disclosure features a method of treating or preventing a disease or condition of the human female reproductive system in a subject in need thereof, the method including administering to the subject a therapeutically effective amount of a candidate pharmaceutical intervention, wherein the candidate pharmaceutical intervention has previously been identified as being efficacious in treating the disease or condition by a method including: (a) contacting the candidate pharmaceutical intervention with the organoid of any one of the foregoing aspects or embodiments of the disclosure; and (b) determining that the organoid exhibits (i) an increase (e.g., an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more) in one or more metrics of ovarian and/or uterine function relative to a measurement of the one or more metrics of ovarian and/or uterine function obtained prior to the contacting, and/or (ii) a decrease (e.g., a decrease of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more) in one or more metrics of severity of the disease or condition relative to a measurement of the one or more metrics of severity of the disease or condition obtained prior to the contacting.

In some embodiments, the subject is a human.

In some embodiments, the disease or condition is one that is associated with a decline in ovarian function. In some embodiments, the condition is a timewise reduction in ovarian function. In some embodiments, the timewise reduction in ovarian function is due to menopause, optionally wherein the menopause is premature menopause. In some embodiments, the decline in ovarian function is a decline in one or more of follicular development, oocyte release, and oocyte maturation.

In some embodiments, the disease is primary ovarian insufficiency (POI), polycystic ovarian syndrome (PCOS), ovarian cysts, ovarian cancer, or ovarian hyperstimulation syndrome. In some embodiments, the disease or condition is one that adversely affects uterine cell function or viability. In some embodiments, the disease or condition is endometriosis, uterine fibroids, adenomyosis, a gynecological cancer, pelvic inflammatory disease (PID), cervical dysplasia, or pelvic floor prolapse. In some embodiments, the disease or condition is one that reduces the subject's fertility. In some embodiments, the disease or condition is embryo implantation failure.

In some embodiments, the candidate pharmaceutical intervention is a small molecule, a polynucleotide (e.g., an antisense oligonucleotide, small interfering ribonucleic acid (siRNA), short hairpin RNA (shRNA), or microRNA (miRNA)), a polypeptide (e.g., an antibody or antigen-binding fragment thereof), a gene therapy (e.g., a DNA or RNA vector, optionally wherein the vector is a viral vector), or a cell therapy (e.g., a chimeric antigen receptor T-cell (CAR-T cell) therapy).

In some embodiments, the one or more metrics of ovarian and/or uterine function include: (a) the rate and/or extent to which the organoid matures an immature oocyte upon co-culturing the organoid with the immature oocyte; (b) the rate and/or extent to which the organoid secretes an estrogen or progestogen, optionally wherein the estrogen is estradiol and/or the progestogen is progesterone; (c) the viability of the organoid or a component cell type thereof; and/or (d) the rate and/or extent to which the organoid specifically binds to one or more cells that model an embryo.

In some embodiments, the immature oocyte is a germinal vesicle (GV)-stage oocyte. In some embodiments, the immature oocyte is a metaphase I (MI)-stage oocyte. In some embodiments, maturation of the immature oocyte is assessed by evaluating conversion of the immature oocyte into a metaphase II (MII)-stage oocyte.

In some embodiments, the one or more metrics of severity of the disease or condition include: (a) abnormal or aberrant growth of the organoid or a component cell type thereof; (b) the extent to which the organoid secretes an estrogen or progestogen above or below a reference level of estrogen or progestogen secretion; and/or (c) the rate at which the organoid or a component cell type thereof undergoes necrosis, apoptosis, or other form of cell death.

In some embodiments, the reference level of estrogen or progestogen secretion is a level of estrogen or progestogen secretion observed in a healthy, pre-menopausal human female subject that does not have the disease or condition. In some embodiments, the estrogen is estradiol. In some embodiments, the reference level of estradiol secretion is a concentration of from about 20 pg/ml to about 50 pg/ml. In some embodiments, the progestogen is progesterone.

In a further aspect, the disclosure features a method of determining whether a candidate pharmaceutical intervention is safe for administration to a female subject having a disease or condition, the method including: (a) contacting the candidate pharmaceutical intervention with the organoid of any one of the foregoing aspects or embodiments of the disclosure; (b) determining that the organoid does not exhibit (i) a decrease (e.g., a decrease of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more) in one or more metrics of ovarian and/or uterine function relative to a measurement of the one or more metrics of ovarian and/or uterine function obtained prior to the contacting, and/or (ii) an increase (e.g., an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more) in one or more indicators of a gynecological disorder relative to a measurement of the one or more metrics of a gynecological disorder obtained prior to the contacting; and, optionally, (c) releasing the candidate pharmaceutical intervention for administration to the subject.

In some embodiments, the method further includes: (d) administering a therapeutically effective amount of the candidate pharmaceutical intervention to the subject to treat the disease or condition.

In another aspect, the disclosure features a method of treating or preventing a disease or condition in a subject (e.g., a female subject) in need thereof, the method including: (a) contacting the candidate pharmaceutical intervention with the organoid of any one of the foregoing aspects or embodiments of the disclosure; (b) determining that the organoid does not exhibit (i) a decrease (e.g., a decrease of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more) in one or more metrics of ovarian and/or uterine function relative to a measurement of the one or more metrics of ovarian and/or uterine function obtained prior to the contacting, and/or (ii) an increase (e.g., an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more) in one or more indicators of a gynecological disorder relative to a measurement of the one or more metrics of a gynecological disorder obtained prior to the contacting; and, optionally, (c) releasing the candidate pharmaceutical intervention for administration to the subject.

In another aspect, the disclosure features a method of treating or preventing a disease or condition in a subject (e.g., a female subject) in need thereof, the method including administering to the subject a therapeutically effective amount of a candidate pharmaceutical intervention, wherein the candidate pharmaceutical intervention has previously been identified as being safe for administration to the subject by a method including: (a) contacting the candidate pharmaceutical intervention with the organoid of any one of the foregoing aspects or embodiments of the disclosure; and (b) determining that the organoid does not exhibit (i) a decrease (e.g., a decrease of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more) in one or more metrics of ovarian and/or uterine function relative to a measurement of the one or more metrics of ovarian and/or uterine function obtained prior to the contacting, and/or (ii) an increase (e.g., an increase of 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, or more) in one or more indicators of a gynecological disorder relative to a measurement of the one or more metrics of a gynecological disorder obtained prior to the contacting.

In some embodiments, the subject is a human.

In some embodiments, the disease or condition is one that is associated with a decline in ovarian function. In some embodiments, the condition is a timewise reduction in ovarian function. In some embodiments, the timewise reduction in ovarian function is due to menopause, optionally wherein the menopause is premature menopause. In some embodiments, the decline in ovarian function is a decline in one or more of follicular development, oocyte release, and oocyte maturation.

In some embodiments, the disease is POI, PCOS, ovarian cysts, ovarian cancer, or ovarian hyperstimulation syndrome. In some embodiments, the disease or condition is one that adversely affects uterine cell function or viability. In some embodiments, the disease or condition is endometriosis, uterine fibroids, adenomyosis, a gynecological cancer, PID, cervical dysplasia, or pelvic floor prolapse. In some embodiments, the disease or condition is one that reduces the subject's fertility. In some embodiments, the disease or condition is embryo implantation failure.

In some embodiments, the candidate pharmaceutical intervention is a small molecule, a polynucleotide (e.g., an antisense oligonucleotide, siRNA, shRNA, or miRNA), a polypeptide (e.g., an antibody or antigen-binding fragment thereof), a gene therapy (e.g., a DNA or RNA vector, optionally wherein the vector is a viral vector), or a cell therapy (e.g., a chimeric antigen receptor T-cell (CAR-T cell) therapy).

In some embodiments, the one or more metrics of ovarian and/or uterine function include: (a) the rate and/or extent to which the organoid matures an immature oocyte upon co-culturing the organoid with the immature oocyte; (b) the rate and/or extent to which the organoid secretes an estrogen or progestogen, optionally wherein the estrogen is estradiol and/or the progestogen is progesterone; (c) the viability of the organoid or a component cell type thereof; and/or (d) the rate and/or extent to which the organoid specifically binds to one or more cells that model an embryo. In some embodiments, the immature oocyte is a germinal vesicle (GV)-stage oocyte. In some embodiments, the immature oocyte is a metaphase I (MI)-stage oocyte. In some embodiments, maturation of the immature oocyte is assessed by evaluating conversion of the immature oocyte into a metaphase II (MII)-stage oocyte.

In some embodiments, the one or more indicators of a gynecological disorder include: (a) abnormal or aberrant growth of the organoid or a component cell type thereof; (b) the extent to which the organoid secretes an estrogen or progestogen above or below a reference level of estrogen or progestogen secretion; and/or (c) the rate at which the organoid or a component cell type thereof undergoes necrosis, apoptosis, or other form of cell death.

In some embodiments, the reference level of estrogen or progestogen secretion is a level of estrogen or progestogen secretion observed in a healthy, pre-menopausal human female subject that does not have the disease or condition. In some embodiments, the estrogen is estradiol. In some embodiments, the reference level of estradiol secretion is a concentration of from about 20 pg/ml to about 50 pg/ml (e.g., 20 pg/ml, 21 pg/ml, 22 pg/ml, 23 pg/ml, 24 pg/ml, 25 pg/ml, 26 pg/ml, 27 pg/ml, 28 pg/ml, 29 pg/ml, 30 pg/ml, 31 pg/ml, 32 pg/ml, 33 pg/ml, 34 pg/ml, 35 pg/ml, 36 pg/ml, 37 pg/ml, 38 pg/ml, 39 pg/ml, 40 pg/ml, 41 pg/ml, 42 pg/ml, 43 pg/ml, 44 pg/ml, 45 pg/ml, 46 pg/ml, 47 pg/ml, 48 pg/ml, 49 pg/ml, or 50 pg/ml). In some embodiments, the progestogen is progesterone.

In further aspect, the disclosure features a platform for engineering a human organoid replica for reproductive screening, the platform including: (a) an engineered reproductive cell; (b) a detector that measures a response by the engineered reproductive cell upon exposure of the engineered reproductive cell to a stimulus; (c) a computing device in communication with the detector, wherein the computing further includes (i) at least a processor and (ii) a memory communicatively connected to the processor, the memory containing instructions configuring the processor to process stimulus data received from the detector and compare the stimulus data to a user profile.

In some embodiments, the engineered reproductive cell is generated by way of bioprinting. In some embodiments, the stimulus relates to drug metabolism. In some embodiments, the stimulus relates to a toxicity screen. In some embodiments, the stimulus relates to a disease model. In some embodiments, the stimulus relates to an epigenetic model.

In some embodiments, processing the stimulus data includes utilizing a cell viability assay. In some embodiments, processing the stimulus data includes utilizing a classifier configured to generate an image-based profile assessment. In some embodiments, comparing the stimulus data to a user profile includes generating a reproductive discrepancy treatment plan.

In some embodiments, the method further includes utilizing the engineered human organoid replica for embryo culture peri-implantation modeling.

In another aspect, the disclosure features a method for engineering a human organoid replica for reproductive screening, the method including: (a) creating a user profile as a function of a reproductive cell relating to a user; (b) receiving a plurality of iPSCs (e.g., hiPSCs); (c) recapitulating, in vitro, the reproductive cell utilizing the plurality of iPSCs; and (d) generating, as a function of the recapitulated reproductive cell, a human organoid replica of an ovary.

In some embodiments, creating the user profile further includes profiling an ovary at a single cell resolution. In some embodiments, profiling the ovary further includes utilizing in silico target discovery.

In some embodiments, creating the user profile further includes identifying at least a reproductive cell from an ovary, wherein the reproductive cell demonstrates a reproductive discrepancy. In some embodiments, the reproductive discrepancy includes a cell health change. In some embodiments, the reproductive discrepancy includes endometriosis.

In some embodiments, recapitulating the reproductive cell further includes replicating a reproductive cell demonstrating a reproductive discrepancy. In some embodiments, recapitulating the reproductive cell further includes utilizing transcription factor-directed cell differentiation.

In some embodiments, generating the human organoid replica further includes generating a human organoid replica containing a reproductive cell demonstrating a reproductive discrepancy. In some embodiments, generating the human organoid replica further includes utilizing bioprinting.

### BRIEF DESCRIPTION OF THE DRAWINGS

The accompanying drawings are included to illustrate embodiments of the disclosure and further an understanding of its implementations.

FIG. 1 is an exemplary embodiment of a platform for engineering a human organoid replica for reproductive screening.

FIG. 2 is an exemplary embodiment of a machine-learning module.

FIG. 3 is an exemplary embodiment of a neural network.

FIG. 4 is a diagram of an exemplary embodiment of a node of a neural network.

FIG. 5 is an exemplary flow diagram of a method for engineering a human organoid replica for reproductive screening.

FIG. 6 is an exemplary diagram of a human organoid replica applied as a disease model.

FIG. 7 is an exemplary diagram of a human organoid replica applied as a druggable disease.

FIG. 8 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes”and “included,”is not limiting.

As used herein, the terms “about” or “approximately” refer to a value that is within 10% (10%, 9%, 8%, 7%, 6%, 5%, 4%,3%, 2%, 1%, or less) above or below the value being described. For instance, the phrase “about 50 mg”refers to a value between and including 45 mg and 55 mg.

As used herein, the term “assisted reproductive technology” or “ART” refers to a fertility treatment in which one or more female gametocytes (oocytes) or gametes (ova) are manipulated ex vivo so as to promote the formation of an embryo that can, in turn, be implanted into a subject in an effort to achieve pregnancy. For example, in some embodiments, a cell or tissue derived from a subject may be manipulated or engineered in vitro using, e.g., co-culturing methodologies described herein. In some embodiments, upon the formation of a mature oocyte (ovum), the ovum may be treated with one or more sperm cells so as to promote the formation of a zygote and, ultimately, an embryo. The embryo may then be transferred to the uterus of a female subject, for instance, using the compositions and methods in the art. Exemplary ART procedures include in vitro fertilization (IVF) and intracytoplasmic sperm injection (ICSI) techniques described herein and known in the art.

As used herein, the terms “subject” refers to an organism that receives treatment for a particular disease or condition as described herein. Examples of subjects and subjects include mammals, such as humans (e.g., a female human), receiving treatment for diseases or conditions that correspond to a reduced ovarian reserve, release of immature oocytes, ovarian decline, or reproductive dysfunction. In some embodiments, a subject refers to an individual receiving treatment or a broader patient population in need of a treatment.

As used herein, “treatment” and “treating” in reference to a disease or condition, refer to an approach for obtaining beneficial or desired results, e.g., clinical or pharmacological results, for reduced ovarian reserve, release of immature oocytes, ovarian decline, or reproductive dysfunction, among other disease indications described herein. A treatment may refer to treating, slowing or inhibiting the onset of, slowing or inhibiting the progression, and/or reversing the progression of a disease or condition. A treatment may refer to alleviating or ameliorating one or more symptoms of a disease or condition. As used herein, treatment or treating may refer to an individual subject or a broader patient population for a particular disease or condition.

As used herein, the terms “therapeutic agent,” “therapeutic intervention,” and “pharmaceutical intervention” are used interchangeably to refer to an agent that, when administered to a subject, has a therapeutic, diagnostic, and/or prophylactic effect and/or elicits a desired biological and/or pharmacological effect.

As used herein, the term “therapeutically effective amount” means an amount of an agent to be delivered (e.g., therapeutic agent) that is sufficient—when administered to a subject suffering from or susceptible to a disease, disorder, and/or condition described herein—to treat, improve the symptoms of, alleviate, ameliorate, and/or delay the onset of the disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

As used herein in the context of a candidate pharmaceutical intervention, candidate therapeutic agent, or the like, the term “release” means to identify the candidate pharmaceutical intervention (or candidate therapeutic agent, or the like) as being efficacious and/or safe for administration to a subject (e.g., a human female subject, such as a subject having a disease or condition described herein) and to permit the intervention or agent (or the like) to be administered to the subject (e.g., for the treatment of a disease or condition described herein).

As used herein in the context of a candidate pharmaceutical intervention, candidate therapeutic agent, or the like, the term “release” means to identify the candidate pharmaceutical intervention (or candidate therapeutic agent, or the like) as being efficacious and/or safe for administration to a subject (e.g., a human female subject, such as a subject having a disease or condition described herein) and to permit the intervention or agent (or the like) to be administered to the subject (e.g., for the treatment of a disease or condition described herein).

As used herein, the term “controlled ovarian hyperstimulation” refers to a procedure in which ovulation is induced in a subject, such as a human subject, prior to oocyte or ovum retrieval for use in embryo formation, for instance, by in vitro fertilization (IVF) or intracytoplasmic sperm injection (ICSI). Controlled ovarian hyperstimulation procedures may involve administration of follicle-stimulating hormone (FSH), human chorionic gonadotropin (hCG), and/or a gonadotropin-releasing hormone (GnRH) antagonist to the subject so as to promote follicular maturation. Controlled ovarian hyperstimulation methods are known in the art and are described herein as they pertain to methods for inducing follicular maturation and ovulation in conjunction with assisted reproductive technology.

As used herein, the term “derived from,” in the context of a cell derived from a subject, refers to a cell, such as a mammalian ovum, that is either isolated from the subject or obtained from expansion, division, maturation, or manipulation (e.g., ex vivo expansion, division, maturation, or manipulation) of one or more cells isolated from the subject. For instance, an ovum is “derived from” a subject or an oocyte as described herein if the ovum is directly isolated from the subject or obtained from the maturation of an oocyte isolated from the subject, such as an oocyte isolated from the subject from about 1 day to about 5 days following the subject receiving ovarian hyperstimulation procedures (e.g., an oocyte isolated from the subject from about 2 days to about 4 days following ovarian hyperstimulation procedures).

As used herein, the term “dose” refers to the quantity of a therapeutic agent, such as a follicle stimulating agent described herein, that is administered to a subject for the treatment of a disorder or condition, such as to enhance oocyte maturation and/or release and promote retrieval and ex vivo maturation of viable oocytes. A therapeutic agent as described herein may be administered in a single dose or in multiple doses. In each case, the therapeutic agent may be administered using one or more unit dosage forms of the therapeutic agent. For instance, a single dose of 100 mg of a therapeutic agent may be administered using, e.g., two 50 mg unit dosage forms of the therapeutic agent. Similarly, a single dose of 300 mg of a therapeutic agent may be administered using, e.g., six 50 mg unit dosage forms of the therapeutic agent or two 50 mg unit dosage forms of the therapeutic agent and one 200 mg unit dosage form of the therapeutic agent, among other combinations. Similarly, a single dose of 900 mg of a therapeutic agent may be administered using, e.g., six 50 mg unit dosage forms of the therapeutic agent and three 200 mg unit dosage forms of the therapeutic agent or ten 50 mg unit dosage form of the therapeutic agent and two 200 mg unit dosage forms of the therapeutic agent, among other combinations.

As used herein, the term “follicle-stimulating hormone” (FSH) refers to a biologically active heterodimeric human fertility hormone capable of inducing ovulation in a subject. FSH may be purified from post-menopausal human urine or produced as a recombinant protein product. Exemplary recombinant FSH products include follitropin alfa (GONAL-F®, Merck Serono/EMD Serono) and follitropin beta (PUREGON™/FOLLISTIM®, MSD/Scherig-Plough).

As used herein, the term “human chorionic gonadotropin” (hCG) refers to the polypeptide hormone that interacts with the luteinizing hormone chorionic gonadotropin receptor (LHCGR) to induce follicle maturation and ovulation. hCG may be purified from the urine of pregnant women or produced as a recombinant protein product. Exemplary recombinant hCG products include choriogonadotropin alfa (OVIDREL®, Merck Serono/EMD Serono).

As used herein, the term “in vitro fertilization” (IVF) refers to a process in which an ovum, such as a human ovum, is contacted ex vivo with one or more sperm cells so as to promote fertilization of the ovum and zygote formation. The ovum can be derived from a subject, such as a human subject, undergoing various ARTs known in the art. For instance, one or more oocytes may be obtained from the subject following injection of follicular maturation stimulating agents for controlled ovarian hyperstimulation procedures, e.g., from about 1 day to about 5 days prior after injection of said agents (such as from about one day to about 4 days after injection of follicular maturation stimulating agents to the subject). The ovum may also be retrieved directly from the subject, for instance, by transvaginal ovum retrieval procedures known in the art.

As used herein, the term “intracytoplasmic sperm injection” (ICSI) refers to a process in which a sperm cell is injected directly into an ovum, such as a human ovum, so as to promote fertilization of the ovum and zygote formation. The sperm cell may be injected into the ovum, for instance, by piercing the oolemma with a microinjector so as to deliver the sperm cell directly to the cytoplasm of the ovum. ICSI procedures useful in conjunction with the compositions and methods described herein are known in the art and are described, for instance, in WO 2013/158658, WO 2008/051620, and WO 2000/009674, among others, the disclosures of which are incorporated herein by reference as they pertain to compositions and methods for performing intracytoplasmic sperm injection.

As used herein, the terms “ovum” and “oocyte” refer to a haploid female reproductive cell or gamete. In the context of assisted reproductive technology as described herein, ova may be produced ex vivo by maturation of one or more oocytes isolated from a subject undergoing ART. Ova may also be isolated directly from the subject, for example, by transvaginal ovum retrieval methods described herein or known in the art. Ovum or oocyte as used in this disclosure may refer to a plurality of oocytes. An oocyte may be in complex with surrounding cells such as a cumulus-oocyte complex (COC).

As used herein, the terms “mature ova” and “mature oocyte” refer to one or more ovum or oocyte in metaphase II (MII)-stage of meiosis and typically has morphological or structural features consistent with metaphase II, such as a polar body and other features described herein.

As used herein, the terms “immature ovum” and “immature oocyte” refer to an ovum or oocyte that has not reached the MII stage of meiosis. In some embodiments, an immature oocyte may be an oocyte including germinal vesicle (GV)-stage and/or metaphase I (MI)-stage oocytes as determined by morphological features and/or other indications described herein and known in the art.

As used herein, the term “oocyte maturation” refers to the process by which an immature oocyte developmentally transitions to a mature oocyte. Oocyte maturation occurs as immature oocytes undergo cell signaling events incurred by external and internal stimuli. External stimuli may be produced by neighboring cells or supporting cells described herein. Oocyte maturation may occur prior to the release of an oocyte and retrieval from a subject. Oocyte maturation may occur following administration of an implant as described herein.

As used herein, an “ovarian support cell” (OSC) or “support cell” refers to one or more cells that promotes maturation of one or more oocytes. An OSC may be an ovarian granulosa cell (e.g., a type of granulosa cell described herein), an ovarian stroma cell (e.g., a type of stroma cell described herein), an ovarian lutein cell (e.g., a type of lutein cell described herein), and/or an ovarian theca cell (e.g., a type of theca cell described herein). An OSC may form a cumulus-oocyte complex (COC) with an oocyte. An OSC may be generated from an exogenous source, such as from induced pluripotent stem cells (iPSCs), e.g., human induced pluripotent stem cells (hiPSCs), as described herein.

An OSC may be applied to a retrieved oocyte using in vitro cell culture methods and compositions described herein. A population of OSCs (an “OSC population”) may be a mixture of two or more cell types. An OSC population may be a mixture of granulosa cells, ovarian stroma cells, lutein cells, and theca cells, e.g., such that the mixture is approximately a 1:1:1:1 population of granulosa cells, stroma cells, lutein cells, and theca cells. An OSC population may be a mixture of granulosa cells, stroma cells, lutein cells, and theca cells such that one cell type is in higher relative abundance compared to one or more cell types (e.g., such that the mixture is approximately a 2:1:1:1 population, a 2:2:2:1 population, a 3:1:1:1 population, a 3:2:2:1 population, a 3:3:3:1 population, a 4:1:1:1 population, a 4:3:2:1 population, a 4:4:2:1 population, a 4:4:4:1 population, a 5:1:1:1 population, a 5:4:3:1 population, a 5:3:2:1 population, a 5:4:2:1 population, or a 5:5:5:1 population of granulosa cells, stroma cells, lutein cells, and theca cells, among other possible population distributions).

An OSC population may be a mixture of granulosa cells, stroma cells, lutein cells, and theca cells such that one cell type is more abundant in the mixture (e.g., 85% granulosa cells, 5% stroma cells, 5% lutein cells, and 5% theca cells; 80% granulosa cells, 10% stroma cells, 5% lutein cells, and 5% theca cells; 70% granulosa cells, 20% stroma cells, 5% lutein cells, and 5% theca cells; 60% granulosa cells, 20% stroma cells, 10% lutein cells, and 10% theca cells; 50% granulosa cells, 10% stroma cells, 10% lutein cells, and 30% theca cells; 40% granulosa cells, 10% stroma cells, 20% lutein cells, and 30% theca cells; 20% granulosa cells, 20% stroma cells, 10% lutein cells, and 50% theca cells; 10% granulosa cells, 10% stroma cells, 50% lutein cells, and 30% theca cells; 10% granulosa, cells, 40% stroma cells, 30% lutein cells, and 20% theca cells, among other possible combinations of cell distributions). In some embodiments, an OSC population may be a mixture of granulosa cells, stroma cells, lutein cells, and theca cells in combination with one or more additional cell types.

An OSC may be genetically manipulated, e.g., using methods known in the art or described herein, to express or overexpress one or more proteins such as transcription factors, enzymes, or secreted hormones. For instance, an OSC may include an engineered granulosa cell configured to express and/or overexpress transcription factor RUNX1. As another example, an engineered ovarian support cell may be engineered to express or overexpress quantities of forms of estrogen, e.g., estrone (E1), estradiol (E2), estriol (E3), estetrol (E4), and/or a combination thereof. An engineered ovarian support cell may overexpress a particular protein and/or transcription factor if a particular protein and/or transcription factor level is detectable at a higher reference range. For example, an engineered ovarian support cell may overexpress a particular protein if the protein is detectable at a level that is 5% higher than the level of the protein expressed from an endogenous naturally occurring polynucleotide encoding the protein.

An OSC may be aggregated with, complexed with, and/or co-cultured with, a human germ cell (e.g., a primordial germ cell like cell (hPGCLC), an oogonia, or an oocyte) to promote or facilitate organoid formation using the methods and compositions described herein.

As used herein, an “ovarian granulosa cell” or a “granulosa cell” is a cumulus cell that has the natural biological function of surrounding an oocyte to ensure healthy oocyte (and subsequent embryo) development. An ovarian granulosa cell may form a COC with an oocyte. An ovarian granulosa cell may express markers consistent with a granulosa subtype such as FOXL2, CD82 and/or follicle-stimulating hormone receptor (FSHR), which can be detected by methods known in the art. An ovarian granulosa cell may be a steroidogenic granulosa cell. An ovarian granulosa cell may be produced from differentiated hiPSCs or otherwise engineered as described herein.

As used herein, a “steroidogenic granulosa cell” is a granulosa cell that may produce one or more steroids, such as estradiol, progesterone, or a combination thereof. The one or more steroids may be produced, e.g., in response to hormonal stimulation, such as by FSH, androstenedione, or a combination thereof.

As used herein, an “ovarian stroma cell” or a “stroma cell” is a cumulus cell that has the natural biological function of surrounding an oocyte to ensure healthy oocyte (and subsequent embryo) development. An ovarian stroma cell may form a COC with an oocyte. An ovarian stroma cell may express markers consistent with a stroma subtype such as nuclear receptor subfamily 2 group F member 2 (NR2F2), which can be detected by methods known in the art. An ovarian stroma cell may be a steroidogenic stroma cell. An ovarian stroma cell may be produced from differentiated hiPSCs, e.g., as described herein.

As used herein, a “steroidogenic stroma cell” is a stroma cell that may produce one or more steroids such as estradiol, progesterone, or a combination thereof. One or more steroids may be produced in response to hormonal stimulation, such as by FSH, androstenedione, or a combination thereof.

As used herein, a “lutein cell” is a cell of the corpus luteum. A lutein cell may be produced, e.g., from differentiated iPSCs (e.g., hiPSCs) or otherwise engineered as described herein.

As used herein, an “ovarian theca cell” or a “theca cell” is an endocrine cell associated with ovarian follicles and that produces one or more androgens and progesterone in the pre-ovulatory large follicles. A theca cell may be produced from differentiated iPSCs (e.g., hiPSCs) or otherwise engineered as described herein.

As used herein, a “germ cell” is a progenitor cell that facilitates sexual reproduction. A germ cell may be formed by in vitro tissue culture methods, such as by way of transcription factor-directed differentiation of iPSCs (e.g., hiPSCs) or other stem cells, using methods described herein. A germ cell may be aggregated with, complexed with, or co-cultured with an OSC (e.g., a granulosa) to form an organoid, such as an ovaroid. A germ cell may originate from a subject (e.g., a subject undergoing IVF or other forms of ART). A germ cell may include, e.g., a primordial germ cell-like cell (hPGCLC), an oogonium, or an oocyte.

As used herein, a “primordial germ cell-like cell (hPGCLC)” is a germ cell precursor that forms during implantation in response to paracrine signaling. An hPGCLC may delineated by the expression of one or more of biomarkers NANOS3, CD38, ITGA6, EpCAM, BLIMP1, TFAP2C, SOX17, or a combination thereof.

As used herein, an “oogonium” is a germ cell that may be found in a human ovary and that forms a primordial follicle with granulosa cells during embryogenesis. An oogonium may be delineated by the expression of one or more of biomarkers DDX4, DAZL, STRA8, or a combination thereof.

As used herein, an “organoid” is a biosynthetic organ that is configured to recapitulate one or more structural or functional properties of a native organ or tissue (e.g., of the female reproductive system). An organoid may be formed by in vitro tissue culture methods, such as by way of transcription factor-directed differentiation of iPSCs (e.g., hiPSCs) or other stem cells, using methods described herein. An organoid may also be formed by bioprinting methodologies, including those described herein and known in the art. Exemplary organoids of the disclosure include those that replicate an ovary or ovarian tissue (i.e., an “ovaroid”), as well as those that replicate a uterus or uterine tissue (i.e., a “uteroid”). Additional examples of organoids include those that replicate other regions of the female reproductive system (e.g., cervical tissue, vaginal tissue, fallopian tubes, or a combination thereof). An organoid, such as an ovaroid or uteroid described herein, may be manipulated genetically, epigenetically, biochemically, or mechanically, to replicate or model a disease state described herein. As is described in the sections below, an organoid may be used as a screening tool for evaluating the treatment efficacy and/or safety of candidate therapeutic interventions for a variety of human pathologies.

As used herein, an “androgen” is any natural or synthetic steroid hormone that regulates the development and maintenance of male characteristics in vertebrates by binding to androgen receptors and are the precursors to estrogens in both males and females. Androgens are natural hormones and are also used as medications in hormone replacement therapy. An androgen may include, e.g., testosterone, dehydroepiandrosterone (DHEA), androstenedione, dehydroepiandrosterone sulfate (DHEA-S), and/or dihydrotestosterone.

As used herein, “progesterone” is a steroid hormone belonging to a class of hormones called progestogens. Physiologically, progesterone prepares the endometrium for the potential of pregnancy after ovulation by triggering the uterine lining to thicken to accept a fertilized egg. Further, progesterone prohibits muscle contractions in the uterus that would cause the body to reject an egg. Low levels of progesterone, in relation to ovarian decline, may cause abnormal menstrual cycles or infertility because the progesterone does not induce an environment conducive for the growth and maturation of a conceived egg.

As used herein, “estrogen” is a category of sex hormone responsible for the development and regulation of the female reproductive system and secondary sex characteristics. Estrogens are required for female fertility and are involved in the maturation and maintenance of the vagina, uterus, ovaries and ovarian function, and ovarian follicles.

As used herein, an “extracellular matrix” is a substrate containing one or more extracellular macromolecules and/or minerals in a network that functions to provide biomechanical structure and/or biochemical support (e.g., signaling cues) to one or more adherent cells.

As used herein, the term “biological sample” or “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, hair, oocyte, ovum, and/or cells isolated from a subject.

As used herein, the term “oral contraceptive treatment,” “oral contraception,” “contraception,” or “birth control pill” refers to a hormonal method of treatment typically used to prevent pregnancy. Oral contraceptive treatment may block the release of oocytes from the ovaries and may contain hormones including estrogen and progestin.

As used herein, the term “ovarian decline” refers to a decline in ovarian function in a subject that typically results in diminished reproductive potential. Ovarian decline may manifest as a reduced ovarian reserve. Ovarian decline naturally occurs with age but may be due to genetic or medical conditions such as, e.g., primary ovarian insufficiency (POI), polycystic ovarian syndrome (PCOS), ovarian cysts, premature menopause, endometriosis, uterine fibroids, gynecological cancer, interstitial cystitis, pelvic inflammatory disease (PID), vaginitis, cervical dysplasia, uterine fibroids, pelvic floor prolapse, interstitial cystitis, among other nonlimiting conditions.

As used herein, the term “ovarian reserve” refers to the number of oocytes in a subject's ovaries and the quality of said oocytes. The ovarian reserve naturally declines with age and/or medical conditions described herein. Subjects with a diminished ovarian reserve may seek IVF or other ARTs to achieve a successful pregnancy. Levels of anti-Mullerian hormone (AMH), as described herein, may be indicative of a subject's ovarian reserve.

As used herein, the terms “follicular triggering agent” or “triggering agent” refer to a chemical or biological composition that stimulates release of oocytes from the ovaries during ovulation. Follicular triggering agents may include hormones such as human chorionic gonadotropin and follicle-stimulating hormone.

As used herein, the term “induced pluripotent stem cells” (iPSCs) refer to artificial stem cells that derive from reprogrammed and otherwise manipulated harvested somatic cells. iPSCs may differentiate into other cell types including ovarian support cells or granulosa cells via methods known in the art and methods described herein. iPSCs may be humans (hiPSCs) or iPSCs from, e.g., other mammalian sources.

As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90% or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure”if it is substantially free of other components.

As used herein, the term “cell culture” refers to laboratory methods that enable in vitro cell proliferation and/or cultivation of a population of cells (e.g., a population of ovarian support cells described herein).

As used herein, the term “co-culture” refers to a type of cell culture method in which more than one cell type or cell populations are cultivated with some degree of contact between them. In a typical co-culture system, two or more cell types may share artificial growth medium.

As used herein, the terms “adherent co-culture systems” or “adherent cell culture” refer to a cell culture arrangement by which cells are attached to a surface for proper growth and proliferation.

As used herein, the terms “suspension co-culture systems” or “suspension cell culture” refer to a cell culture arrangement by which cells are cultivated via dispersion in a liquid medium for proper growth and proliferation.

As used herein, unless otherwise stated, all described concentrations or levels of hormones refer to serum concentrations or levels.

### DETAILED DESCRIPTION

Described herein are compositions and methods for producing organoid models of the human female reproductive system, which can serve as important research tools for screening the safety and efficacy of therapeutic interventions in the assisted reproductive technology (ART) and hormone replacement therapy (HRT) landscapes. For example, the compositions, methods, and apparatuses described herein are directed to organoids of female reproductive organs or tissues (e.g., ovarian organoids (“ovaroids”) and uterine organoids (“uteroids”)), which may be used to evaluate treatments for such conditions as infertility and ovarian decline, among other forms of reproductive dysfunction. The organoids of the disclosure may be used for therapeutic or research purposes, and may be used to assess the safety and/or efficacy of a candidate therapeutic intervention in connection with the treatment of a single patient (e.g., in the context of personalized therapy) or in the context of a broader patient populations (e.g., in connection with the generalized treatment of a class of diseases or conditions described herein).

Without being limited by mechanism, the ovaroid and uteroid models of the disclosure may replicate the structural and/or functional features of endogenous human ovarian and/or uterine tissue by, e.g., containing populations of engineered cell types that recapitulate the antigen expression and/or hormone secretion profiles of their naturally occurring counterparts. For example, the ovaroid models of the disclosure may contain ovarian granulosa, stroma, theca, and/or lutein cells that serve as mimetics for the antigen expression and/or hormone secretion properties of endogenous ovarian granulosa, stroma, theca, and/or lutein cells. Similarly, the uteroid models of the disclosure may contain endometrial, myometrial, and/or perimetrial cells that replicate the antigen expression and hormone secretion properties of endogenous endometrial, myometrial, and/or perimetrial cells. In light of the ability of the organoids described herein to replicate certain properties of their naturally occurring counterparts, the organoids of the disclosure may serve as tools for modeling how endogenous ovarian and/or uterine tissue may react to various stimuli (e.g., to candidate therapeutic interventions). The organoids of the disclosure may, therefore, be used to evaluate the degree to which such candidate interventions are tolerated and/or effective in treating various human disorders.

Exemplary disorders that may be investigated using the organoid models of the disclosure include, without limitation, those that are associated with ovarian decline, such as a timewise reduction in the ovarian reserve (e.g., due to menopause). The compositions, methods, and apparatuses of the disclosure may also be used to investigate therapeutic interventions for diseases that impair ovarian function (e.g., primary ovarian insufficiency (POI), polycystic ovarian syndrome (PCOS), ovarian cysts, premature menopause, endometriosis, uterine fibroids, adenomyosis, gynecological cancers, pelvic inflammatory disease (PID), vaginitis, cervical dysplasia, pelvic floor prolapse, and interstitial cystitis, among other indications described herein).

Accordingly, the compositions, methods, and apparatuses described herein may recapitulate the structure and hormone composition of ovarian or uterine tissue in a healthy or diseased state. The compositions, methods, and apparatuses of the disclosure provide the benefit of being an isolated representation of an organ or cellular niche that can be carefully studied or manipulated in vitro without physiological differences of a model organism or confounding signaling responses that may occur in vivo. As such, the compositions, methods, and apparatus of the disclosure may be useful for directly evaluating ovarian or uterine responses to therapeutic candidates for the treatment of reproductive diseases or evaluating the impact of compounds or compositions on fertility and related physiological processes.

The following sections describe, in further detail, the structural and functional characteristics of the organoids of the disclosure, as well as how these organoids may be used to screen for therapeutic interventions.

### 1. Organoids of Female Reproductive Organs and Tissues

The disclosed organoids may include, e.g., a matrix of cells and proteinaceous components deriving from transcription factor-directed differentiated pluripotent stem cells that are organized to mimic the structure and function of in vivo organs. The organoids, such as an ovaroid, may contain one or more ovarian granulosa cells, stroma cells, lutein cells, and/or theca cells that effectively mimic the structural and hormonal composition of a human ovary. Similarly, a uteroid of the disclosure may contain one or more endometrial, myometrial, or perimetrial cells so as to replicate the structure and/or function of an endogenous human uterus. Organoids replicating female reproductive organs may be used as a relevant in vitro models for various reproductive diseases or conditions.

### A. Cellular Components

In some embodiments, an organoid is configured to replicate the structural and physiological qualities of an ovary or ovarian tissue to form an ovarian organoid or an ovaroid. An ovaroid may comprise an ovarian support cell (OSC) population. An OSC population may include ovarian granulosa cells, ovarian stroma cells, ovarian lutein cells, and/or ovarian theca cells, optionally in combination with one or more additional cell types. The distribution of cells in an ovaroid may depend on or reflect a subject's particular hormonal, biological, or physiological needs or compositions, as determined by a skilled practitioner (e.g., a physician, a clinician, an OB/GYN, a nurse practitioner, or another skilled professional). In some embodiments, an ovaroid comprises an equal or relatively equal distribution of granulosa, ovarian stroma cells, lutein cells, and ovarian theca cells. In some embodiments, an ovaroid comprises a distribution of cells in which one cell type is more abundant than the other cell types in the population. In other embodiments, an ovaroid comprises a distribution of cells in which each cell type is represented at a different relative abundance such that no two cell types have an equivalent relative distribution.

An ovaroid may include one or more granulosa cells. A granulosa cell is a cumulus cell surrounding the oocyte to ensure healthy oocyte and subsequent embryo development. An ovarian granulosa cell may express markers consistent with a granulosa subtype such as FOXL2, CD82 and/or follicle-stimulating hormone receptor (FSHR), which can be detected by methods known in the art. An ovarian granulosa cell may express one or more transcription factors selected from FOXL2, NR5A1, GATA4, RUNX1, RUNX2, or a combination thereof, at higher levels compared to an accepted reference ovarian cell or other cell type known in the art. An ovarian granulosa cell may be a steroidogenic granulosa cell. An ovarian granulosa cell in an ovaroid may secrete estradiol. An ovarian granulosa cell may derive from hiPSCs, e.g., as described herein.

An ovaroid may include one or more ovarian stroma cells. A stroma cell is a cumulus cell surrounding the oocyte to ensure healthy oocyte and subsequent embryo development. An ovarian stroma cell may express markers consistent with a stroma subtype such as nuclear receptor subfamily 2 group F member 2 (NR2F2), which can be detected by methods known in the art. An ovarian stroma cell may be a steroidogenic stroma cell. An ovarian stroma cell may be produced from differentiated hiPSCs, e.g., as described herein.

An ovaroid may include one or more ovarian lutein cells. A lutein cell is a cell of the corpus luteum. In some embodiments, a lutein cell may express one or more of the following markers consistent with the lutein cell subtype such as KRT19, CYP19A1, STAR, CYP17A1, PGR, or a combination thereof. A lutein cell may secrete progesterone. An ovarian lutein cell may derive from hiPSCs, e.g., as described herein.

An ovaroid may include one or more ovarian theca cells. An ovarian theca cell may express higher levels of transcription factors NR2F2 and/or GATA4 compared to an accepted reference ovarian cell or other cell type known in the art. In the ovary, theca cells convert cholesterol to androstenedione, which is the substrate for estradiol production in granulosa cells. An ovarian theca cell in an ovaroid may secrete androstenedione. An ovarian theca cell may derive from hiPSCs, e.g., using methods described herein.

An ovaroid may include one or more human primordial germ cell-like cells (hPGCLCs). An hPGCLC may express higher levels of biomarkers NANOS3, CD38, ITGA6, EpCAM, BLIMP1, TFAP2C and/or SOX17 compared to an accepted reference ovarian cell or other cell type known in the art. An hPGCLC may derive from hiPSCs, e.g., as described herein.

An ovaroid may include one or more oogonia. An oogonium may express higher levels of biomarkers DDX4, DAZL, and/or STRA8 compared to an accepted reference ovarian cell or other cell type known in the art. An oogonia may derive from hiPSCs, e.g., as described herein.

An ovaroid may include one or more oocytes. An oocyte may express higher levels of biomarkers SYCP1, ZP1, ZP2, REC8, LHX8, and/or SOHLH1 compared to an accepted reference ovarian cell or other cell type known in the art. An oocyte may be immature or mature. An oocyte may be harvested from a subject (e.g., a subject with a gynecological or reproductive condition discussed herein, a subject undergoing IVF or another form of ART, a subject by which the ovaroid is modeled, or a healthy female subject). An oocyte may derive from hiPSCs, e.g., as described herein.

In some embodiments, the organoid recapitulates structural and physiological qualities of one or more types of uterine tissue to form a uterine organoid, or a uteroid. In some embodiments, a uteroid comprises endometrial-like cells of an endometrial layer to model the endometrium, the inner-most lining of the uterus that prevents adhesions between opposed walls of the myometrium and maintains patency of the uterine cavity. In some embodiments, a uteroid comprises myometrial-like cells of the uterine myometrium, the smooth muscle tissue of the uterus that mediates uterine contractions and expansion. In other embodiments, a uteroid comprises perimetrial cells of the uterine perimetrium or serosa layer, the outer layer of epithelial cells of the uterus that provides structural support and reduces friction between the uterus and neighboring pelvic organs.

B. Organoids From iPSCs

In some embodiments, the organoids described herein are constructed, at least in part, from iPSCs (e.g., hiPSCs) that are differentiated into various cell types found in native ovarian or uterine tissues. For example, in some embodiments, an ovaroid is derived entirely from hiPSCs. In some embodiments, an ovaroid is derived from iPSCs (e.g., hiPSCs) that are co-cultured with additional cell types of a different origin. In some embodiments, a uteroid is derived entirely from iPSCs (e.g., hiPSCs). In some embodiments, an ovaroid is derived from iPSCs (e.g., hiPSCs) that are aggregated with, complexed with, or co-cultured with additional cell types of a different origin.

Specialized granulosa cells, stroma cells, lutein cells, theca cells, or germ cells (e.g., hPGCLCs, oogonia, or oocytes) utilized in the methods described herein may be created from hiPSCs using transcription factor (TF)-directed protocols. In some embodiments, hiPSCs may be transformed with any one or more plasmids encoding one or more transcription factors. In some embodiments, hiPSCs may be transformed via electroporation, liposome-mediated transformation, viral-mediated gene transfer, among other cell transformation methodologies known in the art. In some embodiments, gene expression of desired transcription factors may be induced in a doxycycline-dependent manner. In some embodiments, transcription factors are constitutively expressed. In some embodiments, a plasmid or expression vector used for reprogramming hiPSCs may have a reporter gene such as a fluorescent protein. In some embodiments, hiPSCs may differentiate into stroma cells with induced expression of transcription factors including GATA4, FOXL2, or a combination thereof. In some embodiments, hiPSCs may differentiate into granulosa with induced expression of transcription factors including FOXL2, NR5A1, GATA4, RUNX1,RUNX2, or a combination thereof. In addition to a combination of one or more transcription factors of FOXL2, NR5A1, GATA4, RUNX1, and/or RUNX2, hiPSCs may differentiate into granulosa via expression of KLF2, TCF21, NR2F2, or a combination thereof. In another embodiment, an hiPSC may be engineered to include an open reading frame encoding one or more proteins selected from zinc finger protein 281 (ZNF281), LIM homeobox 8 (LHX8), spermatogenesis and oogenesis specific basic helix-loop-helix 1 (SOHLH1), distal-less homeobox 5 (DLX5), hematopoietically-expressed homeobox protein (HHEX), folliculogenesis specific BHLH transcription factor (FIGLA), or a combination thereof.

Reprogramming of hiPSCs to granulosa, stroma cells, lutein cells, theca cells, hPGCLCs, oogonia, or oocytes may be determined by genotyping methods known in the art. Reprogramming of hiPSCs to granulosa may be determined by protein expression using any one or more methods known in the art. Differentiation of hiPSCs to granulosa cells may be determined by relative expression of biomarkers typical of a granulosa cell type including AMHR2, CD82, FOXL2, FSHR, IGFBP7, KRT19, STAR, WNT4, or a combination thereof among other granulosa cell biomarkers known in the art. In some embodiments, reprogramming of hiPSCs to granulosa may be determined by production of growth factors and/or hormones including estradiol and progesterone that may adequately support in vitro maturation of retrieved oocyte via paracrine and juxtacrine cell signaling. In some embodiments, the resulting granulosa cells produce estradiol upon stimulation of androstenedione and FSH or forskolin. Differentiation of hIPSCs to hPGCLCs may be determined by relative expression of biomarkers typical of an hPGCLC cell type, including, e.g., NANOS3, CD38, ITGA6, EpCAM, BLIMP1, TFAP2C, SOX17, or a combination thereof (among other hPGCLC cell biomarkers known in the art).

In some embodiments, the cells described herein (e.g., ovarian granulosa cells, among other cell types of the disclosure) may be produced in multiple batches. In some embodiments, the cells may be frozen and thawed prior to co-culture methods. In some embodiments, the cells are freshly differentiated prior to in vitro maturation method. In some embodiments, the cells may be seeded and equilibrated for 2-8 hours (e.g., 2-3 hours, 2-4 hours, 3-4 hours, 4-6 hours, 5-7 hours, 6-8 hours; e.g., 2 hours, 3 hours, 4 hours, 5 hours, 6 hours, 7 hours, 8 hours) before organoid formation or the addition of oocytes for in vitro maturation.

In some embodiments, a subject may donate hiPSCs. In some embodiments, an hiPSC donor may undergo hormone replacement therapy or assisted reproductive technology to treat one or more conditions discussed herein.

### C. Transgenic Cells for Organoid Engineering

Specialized transgenic cells for organoids may be produced using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. For example, CRISPR-based gene editing techniques can be used to introduce, into an iPSC genome, one or more genes encoding for factors that induce differentiation into ovarian support cells (e.g., ovarian granulosa cells) or uterine cell types. These factors include, e.g., FOXL2, NR5A1, GATA4, RUNX1, and RUNX2. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platforms and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of in vitro Transcription (SPRINT). SPRINT can be used to detect a variety of substances, such as metabolites in subject samples or contaminants in environmental samples, with high throughput or with portable point-of-care devices.

### D. Inactive Ingredients

An organoid, such as an ovaroid or uteroid, may comprise inactive ingredients, including extracellular matrix (ECM) components that provide biochemical and biomechanical structure and support for the active cellular components.

In some embodiments, an organoid is embedded into an ECM containing one or more ECM proteins and/or associated molecules, such as binding partners or ions (e.g., divalent ions, such as calcium ions and/or magnesium ions). In some embodiments, an ovaroid comprises ECM proteins, protein polymers, and/or molecules that can be found in connective tissue. In some embodiments, the ECM comprises one or more types of collagen (e.g., fibrillar collagen; e.g., collagen I, II, III, V, XI), one or more epidermal growth factors (EGFs), elastin (e.g., tropoelastin or mature elastin), fibronectin, vitronectin, laminin, among other glycoproteins, cell adhesion proteins, or plant-derived proteins or protein polymers (e.g., alginate).

In some embodiments, the ECM comprises polymers ranging from 500 to 800 μm in size (e.g., from 500 to 550 μm, 550 to 600 μm, 600 to 650 μm, 650 to 700 μm, 700 to 750 μm, or 750 to 800 μm; e.g., 500 μm, 510 μm, 520 μm, 530 μm, 540 μm, 550 μm, 560 μm, 570 μm, 580 μm, 590 μm, 600 μm, 610 μm, 620 μm, 630 μm, 640 μm, 650 μm, 660 μm, 670 μm, 680 μm, 690 μm, 700 μm, 710 μm, 720 μm, 730 μm, 74 μm, 750 μm, 760 μm, 770 μm, 780 μm, 790 μm, or 800 μm). In some embodiments, the ECM of an ovaroid may include proteoglycans, heparan sulfate, chondroitin sulfate, keratan sulfate, hyaluronic acid, elastin, dermatan sulfate, extracellular vesicles, nanoparticles, microparticles, proteins, cell adhesion proteins, proteoglycans, carbohydrate polymers, non-proteoglycan polysaccharides, and/or other forms of substrates one skilled in the art may understand as applicable to embedding and supporting an ovaroid. In some embodiments, one or more ECM components are derived from purified or partially purified animal or plant tissue. In other embodiments, one or more ECM components are derived from in vitro cell culturing conditions.

### II. Methods of Maintaining Organoids and Applications of Use

### A. Cell Culture Conditions

Ovaroids comprising OSCs, such as granulosa cells, stroma cells, lutein cells, theca cells, and/or germ cells optionally derived from iPSCs (e.g., hiPSCs) or transgenic cells (as described above), may be provided as a composition further containing a cell culture media (e.g., IVF, IVM, (e.g., MediCult IVM media), or LAG media). The cell culture media may include human serum albumin (e.g., at about 5-15 mg/mL, e.g., 10 mg/mL), FSH (e.g., at about 70-80 mlU/mL, e.g., 75 mlU/mL), hCG (e.g., at about 95-105 mlU/mL, e.g., 100 mlU/mL), Androstenedione (e.g., at about 495-505 ng/ml, e.g., 500 ng/mL), Doxycycline (e.g., 0.5-1.5μg/mL, e.g., 1 μg/mL) and other compounds such as hyaluronidase and/or dPBS.

### B. Co-Culturing Conditions

In some embodiments of the methods described herein, an organoid, an oocyte, and/or a cell derived from a subject may be combined with a specialized granulosa cell and/or a specialized stroma cell in a co-culture. A “specialized granulosa cell” and a “specialized stroma cell” refers to a cumulus cell surrounding the oocyte to ensure healthy oocyte and embryo development. In some embodiments, the granulosa and/or stroma co-culture cells are sourced from human induced pluripotent stem cells (hiPSCs). As used in this disclosure, a “co-culture” is a cell cultivation set-up, in which two or more different populations of cells are grown with some degree of contact between them. In some embodiments of the method, an organoid may be formed by combining granulosa cells and hPGCLCs in a co-culture at a ratio of from about 20:1 to about 5:1 (e.g., about 15:1, about 12:1, about 10:1, or about 7:1) to then stimulate the gonadal niche for germ cell maturation or other applications.

In some embodiments, steroidogenic granulosa cells, derived from human induced pluripotent stem cells (hiPSCs), may be co-cultured with immature oocytes (COCs), thereby reconstituting the follicular niche in vitro to promote rapid and efficient oocyte maturation in a manner that reinforces oocyte health and developmental competence. As used in this disclosure, a “steroidogenic granulosa cell” is a granulosa cell expressing high levels of steroidogenic enzymes that produce estradiol. For example, a steroidogenic granulosa cell may be a mural granulosa cell extracted from the antral follicle. Applying steroidogenic granulosa cells in the co-cultures of COCs may increase oocyte maturation in vitro after egg/oocyte retrieval, allowing for utilization of all retrieved eggs/oocyte by directly supplying nutrients, raw materials, and mechanical support to oocytes throughout gametogenesis and folliculogenesis. Steroidogenic granulosa cells may grow and perform oocyte maturation of immature COCs in standard IVF and IVM media as described further below. This may increase the overall pool of available, healthy oocytes for use in IVF and reduce the number of ova/oocyte retrieval procedures a user is subjected to.

In some embodiments of the method, a cell culture or an organoid may be formed by combining an immature oocyte or germ cell with a specialized granulosa cell and/or a specialized stroma cell, which is added to mature the oocyte in the cell culture and thus create a COC after extraction of one or more oocytes following the minimal stimulation protocol. In an embodiment, one or more specialized granulosa cells and/or specialized stroma cells may be thawed during a resting period of one or more COCs. In an embodiment, anywhere from between 50,000-150,000 specialized granulosa cells (e.g., 50,000-60,000 cells, 60,000-70,000 cells, 70,000-80,000 cells, 80,000-90,000 cells, 90,000-100,000 cells, 100,000-110,000 cells, 110,000-120,000 cells, 120,000-130,000 cells, 130,000-140,000 cells, or 140,000-150,000 cells; e.g., 50,000 cells, 55,000 cells, 60,000 cells, 65,000 cells, 70,000 cells, 75,000 cells, 80,000 cells, 85,000 cells, 90,000 cells, 95,000 cells, 100,000 cells, 105,000 cells, 110,000 cells, 115,000 cells, 120,000 cells, 125,000 cells, 130,000 cells, 135,000 cells, 140,000 cells, 145,000 cells, or 150,000 cells) may be combined with a COC during culturing. In an embodiment, thawed specialized granulosa cells may be placed into a culture medium prior to COC retrieval, including anywhere from about 24-120 hours beforehand (e.g., about 24-48 hours, about 48-72 hours, about 72-96 hours, about 96-120 hours; e.g., about 24-36 hours, about 30-40 hours, about 36-48 hours, about 48-56 hours, about 56-72 hours, about 72-84 hours, about 80-96 hours, about 90-100 hours about 96-108 hours, about 108-120 hours; e.g., about 24 hours, about 30 hours, about 36 hours, about 42 hours, about 48 hours, about 56 hours, about 60 hours, about 65 hours, about 72 hours, about 78 hours, about 86 hours, about 92 hours, about 96hours, about 102 hours, about 110 hours, about 115 hours, about 120 hours). A COC may be transferred into culture medium containing thawed specialized granulosa cells to form a group culture as described below in more detail. In an embodiment, a group culture may be cultured in an incubator ranging in time from anywhere between 12-48 hours (e.g., 12-16 hours, 12-20 hours, 18-24 hours, 18-36 hours, 24-36hours, 36-48 hours; e.g., 12 hours, 16 hours, 20 hours, 24 hours, 28 hours, 32 hours, 36 hours, 40 hours, 44 hours, 48 hours). The co-culture may be conducted at a biologically suitable temperature, e.g., 37° C.

In some embodiments of the method, a retrieved oocyte, including immature cumulus-oocyte complexes, may be cultured in a group culture. A “group culture” is an extracted COC combined with one or more additional cells. An additional cell may include any cell grown together with an extracted COC. An additional cell may include a specialized stroma cell. An additional cell may include a specialized granulosa cell. In an embodiment, a group culture may be cultured and/or incubated for a particular length of time, such as from between 12-120 hours (e.g., 12-24 hours, 12-36 hours, 24-48 hours, 36-60 hours, 54-72 hours, 68-96 hours, 96-120 hours; e.g., 12 hours, 14 hours, 16 hours, 18 hours, 20 hours, 22 hours, 24 hours, 26 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, 72 hours, 74 hours, 76 hours, 78 hours, 80 hours, 82 hours, 84 hours, 86 hours, 88 hours, 90 hours, 92 hours, 94 hours, 96 hours, 98 hours, 100 hours, 102 hours, 104 hours, 106 hours, 108 hours, 110 hours, 112 hours, 114 hours, 116 hours, 118 hours, or 120 hours). For example, group culturing may include culturing the COCs with a granulosa co-culture as described further below. In some embodiments, group culturing may include culturing a control group of COCs with no co-culture, as described further below. In some embodiments, a user may donate immature oocytes, such as GV-stage and MI-stage oocytes that may be used in medium as part of the group culture to help grow COCs. Oocyte donation may follow an oocyte retrieval process as discussed above. A subject participating in oocyte donation may be different, or the same, from the subject related to the second biological sample containing immature COCs. In some embodiments, an oocyte donation subject may undergo a stimulation protocol as disclosed above.

In some embodiments, the maturity of the oocyte retrieved from the subject may dictate the length of time during which the oocyte is co-cultured with ovarian support cells (e.g., specialized granulosa cells and/or specialized stroma cells). For example, less mature oocytes (e.g., GV oocytes) may require longer co-culturing periods than oocytes at a more advanced stage of meiosis (e.g., MI oocytes).

In some embodiments regarding the culture of oocytes, cell culture media may include LAG media (Medicult, COOPERSURGICAL®). For example, LAG media may be used for the incubation of oocytes and/or COCs post-retrieval from minimal stimulation protocol. For example, a modified-Medicult IVM media may be used as a baseline control during the culturing process. In some embodiments, the cell culture media may include metabolites. For example, the modified-Medicult IVM media may include human serum albumin, FSH, hCG, androstenedione, doxycycline, or any combination thereof. Media may be equilibrated for about 18 to 24 hours (e.g., about 18 hours, about 20 hours, about 22 hours, about 24hours) pre-culture in a standard sterile 37° C. incubator with O₂ (e.g., having a 1-10% O₂ atmosphere, such as 4-8% O₂ or 5-7% O₂, e.g., 6% O₂) and proper CO₂ levels, which are known in the art. Co-cultures and specialized granulosa cell cultures may be adherent cell cultures in cell culture dishes or flasks. Co-cultures and specialized granulosa cell cultures may be suspension cell cultures in cell culture flasks. Cell culture materials and methods include standard sterile cell culturing methods known in the art. Cell morphology and cell viability may be evaluated via one or more established methods known in the art.

In some embodiments, co-culturing is performed in accordance with methods known in the art. For example, a population of ovarian support cells (e.g., ovarian granulosa cells) may be cryopreserved, thawed. In some embodiments, the ovarian support cells are centrifuged to form a cell pellet and are subsequently resuspended in media suitable for in vitro maturation. In some embodiments, the ovarian support cells are centrifuged one or more additional times and, each time, are resuspended in in vitro maturation media. The ovarian support cells may then be co-cultured with an oocyte obtained from the subject undergoing an ART procedure, thereby inducing oocyte maturation.

### C. Omic Data Collection and Analyses of Organoids, Cells, and Culture Media

In some embodiments of the method, culturing of one or more organoids may include an Omics-based analysis. For example, frozen cell lysates and cell culture media may be analyzed for bulk RNA-sequencing, whole genome bisulfite sequencing (WGBS), mass spectrometry-based proteomics and metabolomics. Cell culture media may be utilized for metabolomics analysis to determine changes in molecular content of media following or during organoid formation. This may be utilized to profile dynamic changes in paracrine signaling between granulosa cells, stroma cells, lutein cells, theca cells, and optionally oocytes, among other possible cell types. The data gathered may then be aggregated for downstream analysis for determination of changes in epigenetic state, metabolite presence, and gene expression between different culture conditions and controls.

In some embodiments of the method, an omics-based analysis may include, genomics, proteomics, transcriptomics, pharmacogenomics, epigenomics, microbiomics, lipidomics, glycomics, transcriptomics culturomics, and/or any other omics one skilled in the art would understand as applicable. For example, frozen cell lysates and cell culture mediums may be analyzed for bulk RNA-sequencing, whole genome bisulfite sequencing (WGBS), mass spectrometry-based proteomics and metabolomics.

Cell culture media may be utilized for metabolomics analysis to determine changes in molecular content of media following co-culture compared to pre-culture media controls to profile dynamic changes in paracrine signaling between granulosa cells and oocytes. As the media components are flash frozen, the sample is effectively quenched and amenable to metabolic assessment. The data gathered may then be aggregated for downstream analysis for determination of changes in epigenetic state, metabolite presence, and gene expression between different co-culture conditions and controls.

### D. Applications of Organoids

An organoid that recapitulates the structural and/or functional properties of a female reproductive organ can be used, e.g., for modeling the toxicology and efficacy effects of candidate therapeutic interventions on endogenous human tissues. Moreover, the organoids of the disclosure provide the benefit of being more useful, reliable models of drug tolerability and efficacy as compared to existing in vivo animal models, particularly given the ability to generate organoids from iPSCs (e.g., hiPSCs) that strongly approximate, with a high degree of fidelity, the naturally occurring ovarian and uterine niches.

In addition to serving as close replicas of the human female reproductive organs, the organoid models of the disclosure can provide the additional benefit of facilitating high-throughput screens for the tolerability and efficacy of candidate therapeutic interventions, while minimizing potentially confounding interactions that may otherwise be obtained from an in vivo animal study. Consequently, the organoids of the disclosure provide a unique method of rapidly evaluating the safety and efficacy of candidate interventions.

In some embodiments, an organoid of the disclosure, such as an ovaroid or uteroid, replicates the ovarian structure and/or function of an individual subject. This may be useful, e.g., for the design of personalized treatment plans and/or for evaluating the safety and efficacy of a candidate therapeutic intervention in the context of a personalized therapeutic regimen. In other embodiments, an organoid of the disclosure (such as an ovaroid or uteroid) replicates the ovarian and/or uterine structure and function of a broader population of patients. This latter context is particularly useful in the design of therapeutic regimens that are more broadly applicable, e.g., to classes of patients having like disorders.

### I. Disease Indications

An organoid of the disclosure may be developed to study the effects of therapeutic interventions on a variety of disease states or conditions that may alter ovarian, uterine, or other forms of reproductive function. The compositions and methods of the disclosure may also be used to replicate diseases or conditions that result in impaired ovarian or uterine function (e.g., primary ovarian insufficiency (POI), polycystic ovarian syndrome (PCOS), ovarian cysts, premature menopause, endometriosis, uterine fibroids, gynecological cancer, interstitial cystitis, pelvic inflammatory disease (PID), vaginitis, and cervical dysplasia, among others). In some embodiments, the compositions and methods of the disclosure may be used to study interventions that facilitate in vitro oocyte maturation, oocyte production and release, oocyte or embryo implantation, and other aspects of assisted reproductive technology.

Without being limited by mechanism, the structure and hormone secretion activity of the organoids described herein may recapitulate native ovarian functions in vivo and provide a useful model for replicating ovarian decline in subjects due to a natural, timewise reduction in the ovarian reserve (e.g., due to menopause). In some embodiments, an ovaroid may recapitulate ovarian physiology consistent with underlying diseases or conditions with impairments or abnormalities related to ovulation follicular development, oocyte release, oocyte maturation, or a combination thereof. In some embodiments, an ovaroid may recapitulate ovarian structure or function related to diseases such as POI, PCOS, premature menopause, ovarian cancer, age-related ovarian decline, ovarian hyperstimulation syndrome, among other related conditions. In some embodiments, an ovaroid may have genetic mutations or abnormalities associated with an individual subject or a broader patient population.

Similarly, the structure and hormone secretion activity of the organoids may recapitulate native uterine functions in vivo and provide a useful model for replicating uterine complications identified in subjects or broad patient populations. In some embodiments, a uteroid may recapitulate uterine physiology consistent with underlying diseases or conditions with impairments or abnormalities related to menstruation, embryo implantation, carrying a pregnancy, or giving birth. Such diseases or conditions include uterine fibroids, heavy menstruation, retrograde menstruation, endometriosis, adenomyosis, uterine cancer, pre-eclampsia, Asherman syndrome, miscarriage, among other conditions. In some embodiments, a uteroid may have genetic mutations or abnormalities associated with an individual subject or a broader patient population.

An organoid may be configured to replicate an individual subject's reproductive organ structure or function based on biological sample data from the subject. A subject may be seeking ART, IVF, or HRT interventions. A subject may be between 20 and 45 years old or older. A subject may have a reduced ovarian reserve due to advancing age and/or a genetic or medical condition (e.g., polycystic ovarian syndrome (PCOS)) that leads to a reduced ovarian reserve. A subject may have an ovarian reserve of 20 or fewer oocytes such that a subject has 1 to 5 oocytes, 4 to 10 oocytes, 8 to 16 oocytes, or 15 to 20 oocytes, e.g., the subject has 1 oocyte, 2 oocytes, 3 oocytes, 4 oocytes, 5 oocytes, 6 oocytes, 7 oocytes, 8 oocytes, 9 oocytes, 10 oocytes, 11 oocytes, 12 oocytes, 13 oocytes, 14 oocytes, 15 oocytes, 16 oocytes, 17 oocytes, 18 oocytes, 19 oocytes, or 20 oocytes. A subject may have anti-Mullerian hormone (AMH) levels that are consistent with reduced ovarian reserve. A subject may have their AMH levels measured by a blood test and other methods known in the art. A subject may have AMH levels between 1 and 6 ng/ml (e.g., 1-2 ng/ml, 2-4 ng/ml, or 4-6 ng/ml; e.g., 1 ng/ml, 2 ng/ml, 3 ng/ml, 4 ng/ml, 5 ng/ml, or 6 ng/ml). A subject may have measured estradiol levels between 20 and 50 pg/mL (e.g., 20-30 pg/mL, 25-35 pg/mL, 30-40 pg/mL, 35-45 pg/mL, or 40-50 pg/mL; e.g., 20 pg/mL, 21 pg/mL, 22 pg/mL, 23 pg/mL, 24 pg/mL, 25 pg/mL, 30 pg/mL, 35 pg/mL, 40 pg/mL, 45 pg/mL, or 50 pg/mL). A subject may be using oral contraception.

A physician or skilled practitioner may evaluate a subject for the methods of making organoids, such as ovaroids, by taking a biological sample from the subject. A biological sample may include a laboratory specimen held by a biorepository for research. In some embodiments, a biological sample may include bodily fluids including blood, saliva, urine, semen (seminal fluid), vaginal secretions, cerebrospinal fluid (CSF), synovial fluid, pleural fluid (pleural lavage), pericardial fluid, peritoneal fluid, amniotic fluid, saliva, nasal fluid, optic fluid, gastric fluid, breast milk, cell culture supernatants, one or more oocytes, and the like. A biological sample may include a medical diagnosis, a medical testimonial by a subject and/or a symptomatic complaint, information collected from a wearable device pertaining to a subject and the like. For example, a biological sample may include information obtained from a visit with a medical professional such as a health history. In yet another non-limiting example, a biological sample may include information such as data collected from a wearable device worn by a user and designed to collect information relating to a user's sleep patterns, exercise patterns, and the like. In an embodiment, a biological sample collected at a particular date and/or time of a user's menstrual cycle. For instance, and without limitation, a biological sample may be collected on the second day of a user's menstrual cycle to evaluate one or more hormone levels. The biological sample may be utilized to determine markers of a subject's ovarian reserve that may be measured by a subject's AMH levels and/or other hormone levels or other indications. Other biological samples that may be utilized to determine one or more markers of a subject's overall health include without limitation menstrual cycle progression, and/or monitor circulating hormone levels such as estradiol (E2), luteinizing hormone (LH), follicle-stimulating hormone (FSH), progesterone (P4), estrone (E1), estriol (E3), testosterone, androgens, dehydroepiandrosterone (DHEA), triiodothyronine (T3), tetraiodothyronine (T4), calcitonin, melatonin, insulin, cortisol, human growth hormone (HGH), adrenaline levels, and other hormones.

In some embodiments of the method, the biological sample may be extracted from the user through an extraction device. An “extraction device” is a device and/or tool capable of obtaining, recording and/or ascertaining a measurement associated with a sample. The extraction device may include a needle, syringe, vial, lancet, Evacuated Collection Tubes (ECT), tourniquet, vacuum extraction tube systems, any combination thereof and the like. For example, the extraction device may comprise a butterfly needle set. Data from a biological sample may include measurements, for example, of serum calcium, phosphate, electrolytes, blood urea nitrogen and creatinine, uric acid, and the like.

In an embodiment of the method, biological sample information of a subject may be obtained from an ultrasound. An “ultrasound,” as used in this disclosure, is any procedure that utilizes sound waves to generate one or more images of a user's body. For example, an ultrasound may be utilized to obtain an image of a subject's reproductive organs and/or tissues. In an embodiment, an ultrasound may be performed at a particular time of a subject's menstrual cycle. For example, a subject may receive an ultrasound on day 2 of her cycle and this may be utilized to determine follicle size and/or follicle count.

ii. Therapeutic Screening and Drug Safety Profiles

The organoids, such as ovaroids and uteroids, described herein may be utilized to screen effective therapies or treatment options for individual subjects or patient populations that have a disease or condition described in the preceding section. An organoid that recapitulates a disease state through genetic, epigenetic, biochemical, or mechanical manipulation may be administered a drug or compound (e.g., a small molecule, an antibody or antibody derivative, a peptide, or an oligonucleotide) to evaluate its treatment efficacy, dosing regimen, suitable formulations, pharmacokinetics, or pharmacodynamic profile. Additionally, an organoid may be exposed to a cell-based therapy (e.g., one or more stem cells, such as hematopoietic stem cells, chimeric antigen receptor T cells (CAR-T cells), or other cell therapy known in the art or described herein) to determine treatment efficacy, dosing regimen, targeting or homing strategies, pharmacokinetics, or pharmacodynamic profiles, among other parameters for a candidate pharmaceutical intervention.

A therapeutic screen using the organoids of the disclosure may be used to identify suitable treatment options to improve ovulation, release of oocytes, oocyte maturation, or other processes related to fertility or healthy reproduction. In some embodiments, the efficacy of a potential therapeutic is evaluated based on changes in released oocytes from an ovaroid. In some embodiments, the efficacy of a potential therapeutic is evaluated based on changes in the number of mature oocytes released from the ovaroid. In some embodiments, the efficacy of a potential therapeutic is evaluated based on oocyte fertilization rates.

Alternatively or additionally, a therapeutic screen using the organoids of the disclosure may be used to identify suitable treatment options to improve embryo implantation and/or subsequent embryo development (e.g., to support healthy pregnancy). In some embodiments, the efficacy of a potential therapeutic is evaluated based on changes in embryo implantation or embryo development in a uteroid. In some embodiments, an efficacious treatment promotes improved embryo implantation compared to a reference sample. In some embodiments, the efficacy of a potential therapeutic is evaluated based on embryonic development after implantation in a uteroid. In some embodiments, embryo health and development is measured based on hallmarks observed in different developmental stages. In some embodiments, an embryo is a zygote or a single-celled fertilized egg. In some embodiments, an embryo is a blastocyst and contains a zona pellucida (ZP), trophectoderm (TE), blastocoel (BL), and inner cell mass (ICM), among other developmental signatures.

In some embodiments, the efficacy of a potential therapeutic is evaluated based on changes in ovaroid or uteroid morphology (e.g., size, lipid content, levels of hydration, cell surface structure, glycan content, protein content, among other indications of cell morphology).

In some embodiments, the efficacy of a potential therapeutic is evaluated based on changes in hormone secretion (e.g., estrogens, progestins, androgens) or metabolites in the ovaroid or uteroid media compared to a reference organoid sample that is not contacted with a candidate therapeutic intervention. Changes in hormone secretion may be evaluated by sequencing methods (e.g., mass spectrometry) or hormone-specific detection methods with an antibody or hormone-detecting compound (e.g., enzyme-linked immunosorbent assay (ELISA), Western blot analysis, lateral flow assay, immunoprecipitation, among other detection methods).

In some embodiments, the efficacy of a potential therapeutic is evaluated based on changes in cell populations or cellular distributions in the ovaroid or uteroid as detected by changes in cellular biomarkers (e.g., surface proteins, transcription factors, cell morphology, or broader gene expression profile) compared to a relevant control not administered the therapeutic. Cell populations or distributions may be evaluated by flow cytometry, mass cytometry, microscopy, among other methods known in the art.

In some embodiments, the efficacy of a potential therapeutic is evaluated based on changes in gene expression in an ovaroid or uteroid. Changes in gene expression may be evaluated by RNA-seq, qRT-PCR, next-generation sequencing (NGS) modalities, epigenetic profiling, metabolomic profiling, among other non-limiting methodologies known in the art.

In some embodiments, the efficacy of a potential therapeutic is evaluated based on its cytotoxicity to an organoid. In some embodiments, cytotoxicity is evaluated through cell viability assays, cell proliferation assays, apoptosis assays, autophagy assays, among other non-limiting examples.

In some embodiments, potential treatments or therapeutic options are directed to treat reproductive diseases or conditions related to ovarian or uterine health. In some embodiments, an ovaroid or uteroid therapeutic screen is used to determine suitable treatments for an individual subject with any one or more of the foregoing diseases or conditions. In other embodiments, an ovaroid or uteroid therapeutic screen is used to determine suitable treatment options for a broader disease indication.

In addition to their use as platform for evaluating potential treatment options for diseases or conditions related to reproductive health, organoids such as ovaroids or uteroids may be used to screen the safety profile of novel drugs and compounds for unrelated diseases and their potential impact on fertility and reproductive health. In some embodiments, ovaroids or uteroids may be used to screen novel drugs or compounds based on their effect on ovarian or uterine tissue and their functionality to assess potentially adverse effects on fertility prior to a clinical trial.

### III. Organoid Kits

In some embodiments, the compositions and methods described herein also feature one or more kits. A kit may contain the compositions described herein or other devices related to organoid screening for applications such as drug screening, oocyte retrieval, oocyte maturation, ovarian support cell generation, or embryo implantation. Such kits may be used for therapeutic or research purposes.

### IV. Apparatuses and Associated Methods

At a high level, aspects of the present disclosure are directed to systems and methods for transcription factor-directed differentiation methods to generate human reproductive cell types from pluripotent stem cells. In an embodiment, these cell types can be utilized for high throughput 2D and 3D modeling of the human reproductive axis.

Aspects of the present disclosure can be used to streamline and improve the costly and inefficient drug development process. Exemplary embodiments illustrating aspects of the present disclosure are described below in the context of several specific examples.

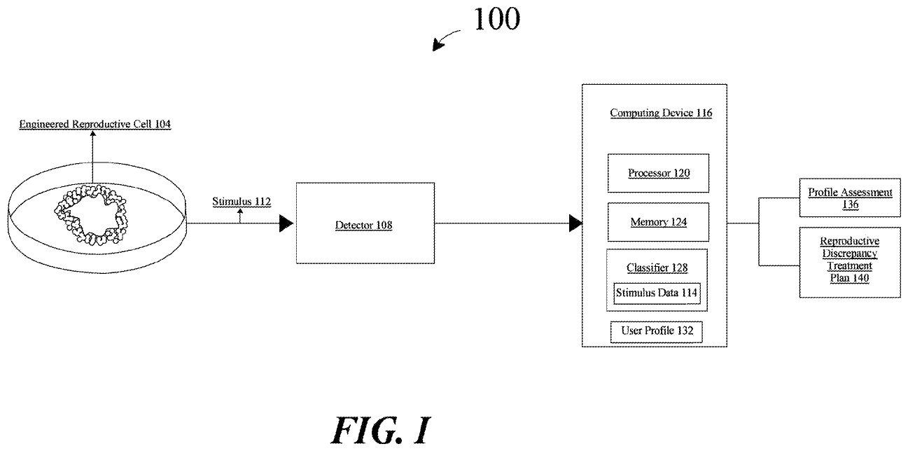

Referring now to FIG. 1 is an exemplary embodiment of a platform 100 for engineering a human organoid replica for reproductive screening. “Reproductive screening,” as used in this disclosure, is the testing of reproductive function and health. A “human organoid,” as used in this disclosure is a structure fabricated in vitro. “In vitro,” as used in this disclosure, is a process performed or taking place outside a living organism. As non-limiting examples, in a test tube, culture dish, and the like. Human organoid replica may be a two-dimensional or three-dimensional tissue like structure derived from stem cells that undergo self-organization and mimic the architecture and functionality of in vivo organs. “Stem cells,” as used in this disclosure, are cells with the potential to develop into many different types of cells in the body. “In vivo,” as used in this disclosure, is a process performed or taking place inside a living organism. A human organoid replica may be derived from pluripotent stem cells as describe further below, adult tissue stem cells, embryonic stem cells (hESCs), induced pluripotent stem cells (hiPSCs), and the like.

In some embodiments, human organoid replicas of an ovary may be generated utilizing bioprinting. “Bioprinting,” as used in this disclosure is the utilization of 3D printing-like techniques to combine cells, growth factors, and/or biomaterials-to fabricate biomedical parts. Bioprinting a human organoid replica may include pre-bioprinting, bioprinting, and post-bioprinting. “Pre-bioprinting,” as used in this disclosure is the process of creating a model that a printer will later create and choosing the materials that will be used. This may include a biopsy of an organ such as an ovary. “Post-bioprinting,” as used in this disclosure, is the process of creating a stable structure from biological material. To maintain structure, both mechanical and chemical stimulations may be used. Stimulations may send signals to the cells to control the remodeling and growth of tissues in the human organoid replica. Methods of bioprinting may include direct printing, coaxial extrusion, indirect, laser, droplet and the like.

Still referring to FIG. 1, bioprinting may be performed without limitation by and/or using an additive manufacturing device. Additive manufacturing devices may include without limitation any device designed or configured to produce a component, organoid, human organoid, human organoid replica, product, or the like using an additive manufacturing process, in which material is deposited on the workpiece to be turned into the finished result. In some embodiments, an additive manufacturing process is a process in which material is added incrementally to a body of material in a series of two or more successive steps. The material may be added in the form of a stack of incremental layers; each layer may represent a cross-section of the object to be formed upon completion of the additive manufacturing process. Each cross-section may, as a non-limiting example be modeled on a computing device as a cross-section of graphical representation of the object to be formed; for instance, a computer aided design (CAD) tool may be used to receive or generate a three-dimensional model of the object to be formed, and a computerized process may derive from that model a series of cross-sectional layers that, when deposited during the additive manufacturing process, together will form the object. The steps performed by an additive manufacturing system to deposit each layer may be guided by a computer aided manufacturing (CAM) tool. In other embodiments, a series of layers are deposited in a substantially radial form, for instance by adding a succession of coatings to the workpiece. Similarly, the material may be added in volumetric increments other than layers, such as by depositing physical voxels in rectilinear or other forms. Additive manufacturing, as used in this disclosure, may specifically include manufacturing done at the atomic and nano level. Additive manufacturing also includes bodies of material that are a hybrid of other types of manufacturing processes, e.g., forging and additive manufacturing as described above. As an example, a forged body of material may have welded material deposited upon it which then comprises an additive manufactured body of material.

Deposition of material in additive manufacturing processes may be accomplished by any suitable means. Deposition may be accomplished using stereolithography, in which successive layers of polymer material are deposited and then caused to bind with previous layers using a curing process such as curing using ultraviolet light. Additive manufacturing processes may include “three-dimensional printing” processes that deposit successive layers of power and binder; the powder may include polymer or ceramic powder, and the binder may cause the powder to adhere, fuse, or otherwise join into a layer of material making up the body of material or product. Additive manufacturing may include metal three-dimensional printing techniques such as laser sintering including direct metal laser sintering (DMLS) or laser powder-bed fusion. Likewise, additive manufacturing may be accomplished by immersion in a solution that deposits layers of material on the body of material, by depositing and sintering materials having melting points such as metals, such as selective laser sintering, by applying fluid or paste-like materials in strips or sheets and then curing that material either by cooling, ultraviolet curing, and the like, any combination of the above methods, or any additional methods that involve depositing successive layers or other increments of material. Methods of additive manufacturing may include without limitation vat polymerization, material jetting, binder jetting, material extrusion, fuse deposition modeling, powder bed fusion, sheet lamination, and directed energy deposition. Methods of additive manufacturing may include adding material in increments of individual atoms, molecules, or other particles. An additive manufacturing process may use a single method of additive manufacturing or combine two or more methods.

Additive manufacturing may include deposition of initial layers on a substrate. A substrate may include, without limitation, a support surface of an additive manufacturing device, or a removable item placed thereon. A substrate may include a base plate, which may be constructed of any suitable material; in some embodiments, where metal additive manufacturing is used, base plate may be constructed of metal, such as titanium. A base plate may be removable. One or more support features may also be used to support additively manufactured body of material during additive manufacture; for instance and without limitation, where a downward-facing surface of additively manufactured body of material is constructed having less than a threshold angle of steepness, support structures may be necessary to support the downward-facing surface; threshold angle may be, for instance 45 degrees. Support structures may be additively constructed and may be supported on support surface and/or on upward-facing surfaces of additively manufactured body of material. Support structures may have any suitable form, including struts, buttresses, mesh, honeycomb or the like; persons skilled in the art, upon reviewing the entirety of this disclosure, will be aware of various forms that support structures may take consistently with the described methods and systems.

Additive manufacturing may alternatively create an element having biological elements, such as an organoid, human organoid, human organoid replica, by printing a support structure having a desired form, followed by deposition of living cells, tissues, or the like thereon. Support structure may be formed of any organic or inorganic material. As a non-limiting example, where organoid, human organoid, human organoid replica is intended to simulate the female reproductive system and/or any portion and/or organ thereof, a support structure may be used to replicate the form and/or structural attributes of pelvic bones, muscular and/or connective tissues surrounding and/or supporting such female reproductive system and/or portion and/or organ thereof, and/or additional portions of the female reproductive system the simulation of which is not necessary for a given application of methods and/or systems described herein. Construction of organoid, human organoid, human organoid replica may alternatively or additional involve use of additive manufacturing to construct a structural support, followed by a non-bioprinting application of biological materials, cell cultures or the like. Additive manufacturing and/or coating processes may alternatively or additionally be used to attach a growth medium, growth matrix, and/or layer of nutrients onto a substrate and/or support structure.

An additive manufacturing device may include an applicator or other additive device. For instance, an additive manufacturing device may include a printer head for a 3D printer. An additive manufacturing device may include an extruding device for extruding fluid or paste material, a sprayer or other applicator for bonding material, an applicator for powering, a sintering device such as a laser, or other such material. An additive manufacturing device may include one or more robotic elements, including without limitation robot arms for moving, rotating, or otherwise positioning a workpiece, or for positioning a manufacturing tool, printer heads, or the like to work on workpiece. An additive manufacturing device may include one or more workpiece transport elements for moving a workpiece or finished part or component from one manufacturing stage to another; workpiece transport elements may include conveyors such as screw conveyors or conveyor belts, hoppers, rollers, or other items for moving an object from one place to another.

Still referring to FIG. 1, platform 100 includes an engineered reproductive cell 104. An “engineered reproductive cell,” as used in this disclosure, is a cell originating from a reproductive system engineered in vitro. Engineered reproductive cell 104 may include any type of cell, tissue, organ, and the like involved in a living organism's reproductive system. For example, engineered reproductive cell 104 may include an ovarian support cell such as a granulosa or theca cell, or a germline cell such as a gamete cell sourced from a sperm and/or egg. In yet another non-limiting example, engineered reproductive cell 104 may include an ovarian germ cell such as a non-gamete cell of the ovary and/or testis. In yet another non-limiting example, engineered reproductive cell 104 may include a mixture of one or more cells. “Engineering,” as used in this disclosure, is a process that alters and/or reproduces the genetic makeup of an organism. Engineered reproductive cell 104 may include one or more cells, tissue samples, a model of a reproductive system/organ/cell (e.g., an ovary, testicles, uterus, scrotum, etc.) as described further below. Engineering a reproductive cell 104 may include replicating genetic makeup and functionality of a received reproductive cell 104, organ, and/or system. For example, engineered reproductive cell 104 may be modeled after received reproductive cells, organs, and/or systems from a plurality of donors across various age groups. For example, replicating received ovarian cells may include analyzing and identifying discrepancies among the cells that may be engineered to generate an accurate replica in vitro representation. In an embodiment, engineered reproductive cell 104 may include a cell originating from a reproductive system in vivo.

Still referring to FIG. 1, in some embodiments, engineering may include altering the DNA makeup such as for example by changing a single base pair, deleting a region of DNA, adding a new segment of DNA, manipulating DNA, modifying DNA, recombining DNA and/or a nucleic acid, and the like. Engineering may include the design and construction of new biological entities such as with the use of laboratory technologies such as enzymes, genetic circuits, and cells or the redesign of existing biological systems. Engineering may include differentiating an engineered reproductive cell 104 to express one or more transcription factors, this process is referred to as transcription factor-directed cell differentiation throughout this disclosure. A “transcription factor,” as used in this disclosure is any protein that controls a rate of transcription. For example, a transcription factor may be selected from NR5A1 and a RUNX family protein. For instance, an engineered reproductive cell 104 may include an engineered granulosa cell configured to express and/or overexpress RUNX 1. An engineered reproductive cell 104 may express a particular protein and/or transcription factor if a level of the protein is detectable such as for example using a known protein assay. An engineered reproductive cell 104 may overexpress a particular protein and/or transcription factor if a particular protein and/or transcription factor level is detectable at a higher reference range. For example, an engineered reproductive cell 104 may overexpress a particular protein if the protein is detectable at a level that is 5% higher than the level of the protein expressed from an endogenous naturally occurring polynucleotide encoding the protein.

Engineering may include engineering one or more polynucleotides of an engineered reproductive cell 104. An “engineered polynucleotide,” as used in this disclosure, is a nucleic acid that does not occur in nature. An engineered polynucleotide may include a recombinant nucleic acid. A “recombinant nucleic acid,” as used in this disclosure, is a molecule that is constructed by joining nucleic acids from two different organisms. For example, a recombinant nucleic acid may be created from a human and a mouse. An engineered polynucleotide may include a synthetic nucleic acid. A “synthetic nucleic acid,” as used in this disclosure, is a molecule that is amplified and/or chemically synthesized. For example, a synthetic nucleic acid may include a chemically modified and/or otherwise modified nucleic acid that can bind to one or more naturally occurring molecules. An engineered polynucleotide may include DNA (genomic DNA, cDNA, and/or any combination thereof), RNA, and/or a hybrid molecule. An engineered polynucleotide may include complementary DNA which may be synthesized from a single stranded RNA (messenger RNA (mRNA) or microRNA (miRNA)) such as for example using a catalyst such as but not limited to reverse transcriptase. In an embodiment, an engineered polynucleotide may include a promoter operably linked to an open reading frame. A “promoter,” as used in this disclosure, is a nucleotide sequence to which RNA polymerase binds to initiate transcription. A promoter may include an inducible promoter. An inducible promoter may be regulated in vitro by a stimulus 112 such as a chemical agent, temperature, or light. This may allow for temporal and/or spatial control of gene expression. For example, an inducible promoter may include but is not limited to an alcohol regulated promoter, a tetracycline operator sequence, a steroid regulated promoter, a human estrogen receptor, and the like.

In an embodiment, engineering may include altering of the cell's ability to express, overexpress and/or secrete a hormone including but not limited to hormones such as estrogen, progesterone, testosterone, DHEA and the like. An engineered reproductive cell 104 may include but is not limited to an engineered granulosa cell, an engineered lutein cell, and/or an engineered theca cell as described below in more detail. In an embodiment, an engineered reproductive cell 104 may be sourced from an oocyte, granulosa cell, cumulus oocyte complex, and similar cells originating in the ovary. An “oocyte,” as used in this disclosure, is a female gametocyte or germ cell involved in reproduction. In an embodiment, an engineered reproductive cell 104 may include an engineered granulosa cell. A “granulosa cell” is an estrogen-secreting cell of the epithelial lining of a graafian follicle and/or or its follicular precursor. Engineering may include any engineering process as described herein. For instance, and without limitation, a granulosa cell may be engineered to overexpress quantities of estradiol. “Estrogen” as used in this disclosure is a steroid hormone that promotes the development and/or maintenance of female sex characteristics. A “cumulus oocyte complex,” as used in this disclosure, is an oocyte containing one or more surrounding cumulus cells. A COC may contain an immature oocyte. A COC may contain a mature oocyte. A “mature oocyte” as used in this disclosure, is one or more mature reproductive cell 104s originating in the ovaries. In some embodiments, engineered reproductive cell 104 may include but is not limited to an engineered cell and/or any combination thereof including oogonia cells, oogonia-like pluripotent stem cells, polynucleotides, primordial germ cells, and primordial germ cell-like pluripotent stem cells. For example, engineered reproductive cell 104 may include a pluripotent stem cell (PSC) incorporating: an engineered polynucleotide including an open reading frame encoding a protein selected from Zinc Finger Protein 281 (ZNF281), LIM Homeobox 8 (LHX8), and Spermatogenesis and Oogenesis Specific Basic Helix-Loop-Helix 1 (SOHLH1). In yet another non-limiting example, engineered reproductive cell 104 may include a pluripotent stem cell (PSC) incorporating an engineered polynucleotide including an open reading frame encoding a protein selected from Distal-Less Homeobox 5 (DLX5), Hematopoietically-expressed homeobox protein (HHEX), and Folliculogenesis Specific BHLH Transcription Factor (FIGLA). In yet another non-limiting example, engineered reproductive cell 104 may include a pluripotent stem cell (PSC) incorporating an engineered polynucleotide including an open reading frame encoding a protein selected from nuclear receptor subfamily 5 group A member 1 (NR5A1) and a Runt-related transcription factor (RUNX) family protein.

Still referring to FIG. 1, in an embodiment, a reproductive cell 104 may be engineered using Clustered Regularly Interspaced Short Palindromic Repeats (CRISPR) technology. “CRISPR” is programmable technology that targets specific stretches of genetic code to edit DNA at precise locations. CRISPR technology may include CRISPR-CAS 9. Cas9 (or “CRISPR-associated protein 9”) is an enzyme that uses CRISPR sequences as a guide to recognize and cleave specific strands of DNA that are complementary to the CRISPR sequence. Cas9 enzymes together with CRISPR sequences form the basis of a technology known as CRISPR-Cas9 that can be used to edit genes within organisms. CRISPR technology may include Class 1 CRISPR systems including type I (cas3), type III (cas10), and type IV and 12 subtypes. CRISPR technology may include Class 2 CRISPR systems including type II (cas9), type V (cas12), type VI (cas13), and 9 subtypes. In some embodiments, CRISPR technology may involve CRISPR-Cas design tools which are computer software platform 100s and bioinformatics tools used to facilitate the design of guide RNAs (gRNAs) for use with the CRISPR/Cas gene editing system. For example, CRISPR-Cas design tools may include: CRISPRon, CRISPRoff, Invitrogen TrueDesign Genome Editor, Breaking-Cas, Cas-OFFinder, CASTING, CRISPy, CCTop, CHOPCHOP, CRISPOR, sgRNA Designer, Synthego Design Tool, and the like. CRISPR technology may also be used as a diagnostic tool. For example, CRISPR-based diagnostics may be coupled to enzymatic processes, such as SHERLOCK-based Profiling of IN vitro Transcription (SPRINT). SPRINT can be used to detect a variety of substances, such as metabolites in patient samples or contaminants in environmental samples, with high throughput or with portable point-of-care devices.

Still referring to FIG. 1, in some embodiments, engineered reproductive cell 104 may be derived from a pluripotent stem cell. “Pluripotent stem cells,” as used in this disclosure, are cells that are able to self-renew by dividing and developing into the three primary groups of cells that make up a human body, including ectoderm, giving rise to the skin and nervous system; endoderm, forming the gastrointestinal and respiratory tracts, endocrine glands, liver, and pancreas; and mesoderm, forming bone, cartilage, most of the circulatory system, muscles, connective tissue, and more. Pluripotent stem cells may be able to make cells from all three of these basic body layers, so they can potentially produce any cell or tissue the body needs to repair itself. Pluripotent stem cells may include induced pluripotent stem cells (iPSCs), which are derived from skin or blood cells that have been reprogrammed back into an embryonic-like pluripotent state that may enable the development of an unlimited source of any type of human cell needed for therapeutic purposes. For example, iPSC can be prodded into becoming beta islet cells to treat diabetes, blood cells to create new blood free of cancer cells for a leukemia patient, or neurons to treat neurological disorders. Induced pluripotent cells may be derived from embryos, embryonic stem cells made by somatic cell nuclear transfer (ntESCs) and/or an embryonic stem cell from an unfertilized egg. In an embodiment, a pluripotent cell may include a human pluripotent cell. In an embodiment, a pluripotent cell may include an embryonic stem cell, such as a human embryonic stem cell. An “embryonic stem cell,” as used in this disclosure, is a pluripotent stem cell made using embryos or eggs. An embryonic stem cell may include but is not limited to a true embryonic stem cell, a nuclear transfer embryonic stem cell, and/or a parthenogenetic embryonic stem cell. In an embodiment, a pluripotent stem cell may include an induced pluripotent stem cell such as a human induced pluripotent stem cell. A human induced pluripotent stem cell may be derived from skin or blood cells that may be engineered back into an embryonic-like pluripotent state that enables the development of an unlimited source of any type of human cell. In some embodiments, engineered reproductive cell 104 may include an engineered a theca cell. A “theca cell,” as used in this disclosure, is one or more endocrine cells associated with ovarian follicles that produce androgens. Engineering may include any engineering process as described herein. In some embodiments, the engineered cell may be a lutein cell. A “lutein cell,” as used in this disclosure, is a cell of the corpus luteum. Engineering may include any engineering process as described herein.

Still referring to FIG. 1, engineering a reproductive cell 104 may include utilizing in silico target discovery. “In silico,” as used in this disclosure, are biological models developed on a computer to model a pharmacologic or physiologic process using computer simulation. In silico discovery of potential biological targets for chemical compounds may offer an alternative avenue for the exploration of ligand-target interactions and biochemical mechanisms, as well as for investigation of drug repurposing. A “biological target” as used in this disclosure, is anything within a living organism to which some other entity (like an endogenous ligand or a drug) is directed and/or binds, resulting in a change in its behavior or function. Examples of common classes of biological targets may include proteins and nucleic acids. For example, in silico discovery techniques may be used to model a reproductive cell 104 for drug and disease testing. In some embodiments, a biological target may be a native protein in the body whose activity is modified by a drug resulting in a specific effect, which may be a desirable therapeutic effect or an unwanted adverse effect. In some embodiments, in silico discovery may be used to model a reproductive cell 104 to discover transcriptomic and proteomic signatures to replicate in human organoid replica. In silico target discovery may include computational target fishing mines biologically annotated chemical databases and then maps compound structures into chemogenomic space to predict the biological targets. Applications in computational target fishing may include chemical similarity searching, data mining/machine learning, panel docking, and the bioactivity spectral analysis for target identification. In some embodiments, engineering a reproductive cell 104 may include utilizing microfluidics. “Microfluidics,” as used in this disclosure, is the behavior, precise control, and manipulation of fluids that are geometrically constrained to a small scale (typically sub-millimeter) at which surface forces dominate volumetric forces. For example, microfluidics for cell biology may be a mini cell culture system where a single cell or a few cells are seeded into a device with input and output channels. These cells may be exposed to dynamic fluid flow, accompanied by live imaging. Microfluidics systems may be used in capillary electrophoresis, isoelectric focusing, immunoassays, flow cytometry, optimization of protein drugs production, sample injection in mass spectrometry, PCR amplification, DNA analysis, separation and manipulation of cells, and cell patterning of a received ovarian cell/and or engineered reproductive cell 104.

Still referring to FIG. 1, platform 100 includes a detector 108 that measures a response by the engineered reproductive cell 104 upon exposure of the engineered reproductive cell 104 to a stimulus 112. A “stimulus,” as used in thus disclosure, is an element that triggers a physical or functional change. A “detector,” as used in this disclosure is a sensor capable measuring a cell response. For example, detector 108 may include detectors that utilize liquid chromatography, mass spectrometer, assays, flow cytometer, and the like. In some embodiments, stimulus 112 may relate to drug metabolism. “Drug metabolism,” as used in this disclosure, is the metabolic breakdown of drugs in cells. The metabolism of pharmaceutical drugs may be an important aspect of pharmacology and medicine. For example, the rate of metabolism determines the duration and intensity of a drug's pharmacologic action. In some embodiments, a human organoid replica may be used for preclinical studies for all products in women's health. By testing drug products on human organoid replica, the success rate of a product may be predicted, tested and/or fine-tuned before clinical and clinical trials. For example, engineered reproductive cell 104 replicating a lutein cell producing low estrogen levels may be stimulate with drugs products configured to increase estrogen production. Detector 108 may then be used to measure the response of engineered reproductive cell 104 to stimulus 112. Data collected from detector 108 may be stored by a computing device 116 and processed as described further below. In some embodiments, stimulus 112 may relate to a toxicity screening. A “toxicity screening,” as used in this disclosure, is a test to identify an element that has a deleterious effect on cell functions and development. An element may include a drug or common molecule. For example, an element may be drugs deleterious to the health of ovarian cells, such as cyclophosphamide, cisplatin and doxorubicin, which may cause premature ovarian insufficiency by inducing death and/or accelerated activation of primordial follicles and increased atresia of growing follicles. In some embodiments, human organoid replica may be used to screen drugs entering clinical trials to ensure the drugs are not toxic to the ovaries and thus allow more woman to safely enroll in drug related trials.

Still referring to FIG. 1, in some embodiments, stimulus 112 may relate to a disease model. A “disease model,” as used in this disclosure, is a cell displaying a pathological process that is observed in at least an actual human disease. For example, human organoid replicas may model an ovary experiencing endometriosis. There may be few to no targeted treatments for endometriosis currently and despite interest from pharmaceutical companies, there may be few to no effective models for endometriosis drug development. Human organoid replica may provide an accurate disease model of endometriosis for drug/treatment development, medicinal studies, and the like. In some embodiments, stimulus 112 may relate to an epigenetic model. An “epigenetic model,” as used in this disclosure, is an organoid used to study heritable phenotype changes that do not involve alterations in the DNA sequence. Epigenetic changes in human organoid replica may include DNA Methylation, Histone modification, Non-coding RNA, and similar changes. For example, epigenetic changes in human organoid replica may be analyzed to determine the effects of anti-aging interventions. The effects of anti-aging interventions may be measured in a shorter time frame than in vivo organs. As a disease model, therapeutics for ovarian reserve preservation and oocyte improvement may tested. For example, peptide AMH mimetics as AMHR2 agonists to fine-tune follicle recruitment and prevent premature ovarian failure and early-onset menopause. Peptide mimics of AMH can fine-tune AMHR2 signaling and prevent over-recruitment of primary follicles, thus preserving the ovarian reserve.

Still referring to FIG. 1, platform 100 includes a computing device 116. Computing device 116 may include any computing device as described in this disclosure, including without limitation a microcontroller, microprocessor 120, digital signal processor 120 (DSP) and/or system on a chip (SoC) as described in this disclosure. Computing device 116 includes a processor 120 and a memory 124 communicatively connected to the processor 120, wherein memory 124 contains instructions configuring processor 120 to process and compare data related to a stimulus response. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled”may be used in place of communicatively connected in this disclosure.

Computing device 116 may include, be included in, and/or communicate with a mobile device such as a mobile telephone or smartphone. Computing device 116 may include a single computing device 116 operating independently or may include two or more computing device 116 operating in concert, in parallel, sequentially or the like; two or more computing devices 116s may be included together in a single computing device 116 or in two or more computing devices 116. Computing device 116 may interface or communicate with one or more additional devices as described below in further detail via a network interface device. Network interface device may be utilized for connecting computing device 116 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices 116, and any combinations thereof. A network may employ a wired and/or a wireless mode of communication. In general, any network topology may be used. Information (e.g., data, software etc.) may be communicated to and/or from a computer and/or a computing device 116. Computing device 116 may include but is not limited to, for example, a computing device 116 or cluster of computing devices 116 in a first location and a second computing device 116 or cluster of computing devices 116 in a second location. Computing device 116 may include one or more computing devices 116 dedicated to data storage, security, distribution of traffic for load balancing, and the like. Computing device 116 may distribute one or more computing tasks as described below across a plurality of computing devices 116 of computing device 116, which may operate in parallel, in series, redundantly, or in any other manner used for distribution of tasks or memory 124 between computing devices 116. Computing device 116 may be implemented using a “shared nothing” architecture in which data is cached at the worker, in an embodiment, this may enable scalability of system 100 and/or computing device 116.

With continued reference to FIG. 1, computing device 116 may be designed and/or configured to perform any method, method step, or sequence of method steps in any embodiment described in this disclosure, in any order and with any degree of repetition. For instance, computing device 116 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 116 may perform any step or sequence of steps as described in this disclosure in parallel, such as simultaneously and/or substantially simultaneously performing a step two or more times using two or more parallel threads, processor 120 cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 116 is configured to process stimulus data 114. Processing data may include analyzing engineered reproductive cell 104 response to stimulus 112 to determine cell health changes, metabolic change, expression changes, and/or genome changes. “Cell health changes,”as used in this disclosure, are changes in the wellness and function of a cell. Determining cell health changes may include utilizing cell viability assays, cytotoxicity assays, apoptosis assays, autophagic assays, autophagic flux, ADME assays, and the like. “Cell viability assays,” as used in this disclosure, are assays created to determine the ability of cells to maintain a state of survival. This may include the ability of cells to recover a state of survival. Determining metabolic changes may include utilizing dinucleotide assays, energy metabolite assays, oxidative stress assays, and the like. Determining expression changes may include utilizing RNA isolation, dye-based/probe-based qPCR and RT-qPCR reagents, promoter analysis receptor assays, and the like. Determining genome changes may include utilizing STR profiling for cell line identity, NGS/Directed Sequencing/SNP, genotyping, epigenetic profile, and the like. In some embodiments, computing device 116 may be configured to process data using a machine algorithm such as a classifier 128. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. classifier 128 may be configured to output at least a datum that labels or otherwise identifies a set of data that are clustered together, found to be close under a distance metric as described below, or the like. Computing device 116 and/or another device may generate classifier 128 using a classification algorithm, defined as a process whereby computing device 116 derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 1, classifier 128 may be used for image-based profile assessment 136 of engineered reproductive cell 104 response to stimuli. A “profile assessment,” as used in this disclosure is a cell evaluation measuring a large number of features in a cell. For example, area, shape, intensity, texture, and other aspects. A profile assessment may utilize assays as described above. A computing device may be configured to analyze microscopy image formats. Additionally computing device may be configured to perform microscopy image processing as part of image-based profile assessment 136. “Microscopy image processing,” as used in this disclosure, is the use of digital image processing techniques to process, analyze and present images obtained from a microscope. An image-based profiling assessment may include assays assessing single-cell phenotypes may be used to explore mechanisms of action, target efficacy and toxicity of small molecules. For example, classifier 128 may receive culture images from detector 108 wherein classifier 128 is configured to generate a phenotypic profiling of reproductive cell 104. Computing device 116 may train classifier 128 using training data including images, labels and descriptions of cells or cell lines representing a plurality of cell morphology in response to a plurality of different stimuli. Training data may contain images of cell samples of various age groups. For example, training data may contain fetal cell images in response acitretin. Training data may contain images of cells in response to specific stimulus 112 dosing. For example, images of cells exposed to 10 mg of cetirizine. In some embodiments, classifier 128 may be configured to perform scoring methods, such as total oocyte scoring as described in U.S. Nonprovisional application Ser. No. 17/846,725, filed on Jun. 22, 2022, and entitled “An Apparatus and Method For Inducing Human Oocyte Maturation In Vitro,”the entirety of which is incorporated herein by reference.

Still referring to FIG. 1, computing device 116 may be configured to generate classifier 128 using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)+P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device 116 may then calculate a likelihood table by calculating probabilities of different data entries and classification labels. Computing device 116 may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 1, computing device 116 may be configured to generate classifier 128 using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors' algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 1, generating k-nearest neighbors'algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute I as derived using a Pythagorean norm: l=n i=O αi2, where ai is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

Still referring to FIG. 1, platform 100 includes a computing device 116 configured to compare stimulus data 114 to a user profile 132. “Stimulus data,” as used in this disclosure, is cell response to a stimulus received from a detector. A “user profile,” as used in this disclosure is a data structure containing analytical data relating to a user. The user profile 132 may contain biological information related to the user such as age, race, family genetics, reproductive cell 104s, and the like. For example, a user profile 132 may include an ovarian disease such as polycystic ovary syndrome being a familial condition related to the user and the likely of the user experiencing the condition. A “user,” as used in this disclosure is a person. Computing devices may generate a classifier 128 as defined above to output a reproductive discrepancy treatment plan 140 for a user. A “reproductive discrepancy treatment plan,” as used in this disclosure is a treatment plan aimed to mitigate a reproductive discrepancy. A “treatment plan” as used in this disclosure, is a detailed proposal for the treatment of a condition. In some embodiments, a reproductive discrepancy treatment plan 140 may be a detailed plan with information about a user's disease, the goal of treatment, the treatment options for the disease and possible side effects, and the expected length of treatment. For example, a reproductive discrepancy treatment plan 140 may be a treatment plan 140 with the goal of countering infertility, hirsutism, acne and/or obesity issues caused by polycystic ovary syndrome. Training data for the classifier 128 may include correlations between user profile 132, stimulus data 114, and data retrieved from a treatment database. A “treatment database,” as used in this disclosure is a data structure containing a plurality of information relating to reproductive cell 104 health, the reproductive system, historical methods of reproductive discrepancy treatment, experimental methods of reproductive discrepancy treatment, side effects associated with treatment plans 140, side effects of medications, and treatment plans 140 associated with certain qualities of a user (e.g., genetics, medical history) and the like. All databases described throughput this disclosure may be communicatively connected to computing device 116 and may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure. Comparison may be performed using any suitable algorithm, including without limitation a classifier, which may include any type of classifier described herein; classifier may be trained using training examples correlating user profiles to stimulus data, which training examples may be entered by users, collected using previous iterations of methods described herein, or the like.

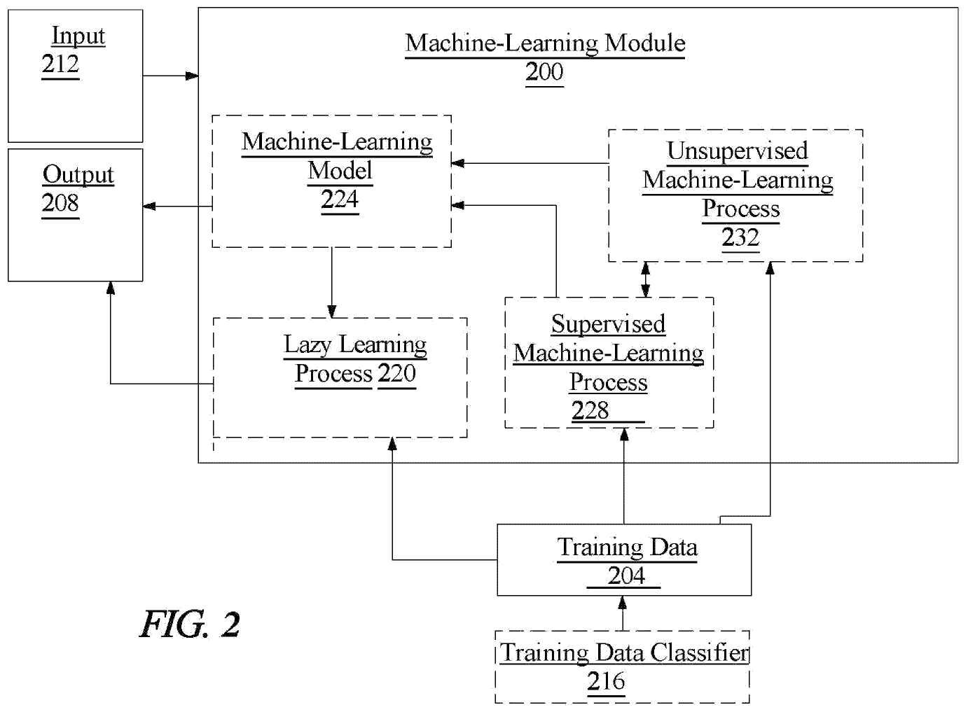

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a user and written in a programming language. Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail below. Training data 204 used by machine-learning module 200 may correlate any input data as described in this disclosure to any output data as described in this disclosure.

Further referring to FIG. 2, training data may be filtered, sorted, and/or selected using one or more supervised and/or unsupervised machine-learning processes and/or models as described in further detail below; such models may include without limitation a training data classifier 216. Training data classifier 216 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a processes whereby a computing device and/or any module and/or component operating thereon derives a classifier from training data 204. Classification may be performed using, without limitation, linear classifiers such as without limitation logistic regression and/or naïve Bayes classifiers, nearest neighbor classifiers such as k-nearest neighbors'classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements. Lazy learning may implement any suitable lazy learning algorithm, including without limitation a K-nearest neighbors'algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs and outputs, as described above, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning processes 232. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the LASSO model is the Frobenius norm amounting to the square root of the sum of squares of all terms. Linear regression models may include the elastic net model, a multi-task elastic net model, a least angle regression model, a LARS LASSO model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g., a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithms may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors'algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

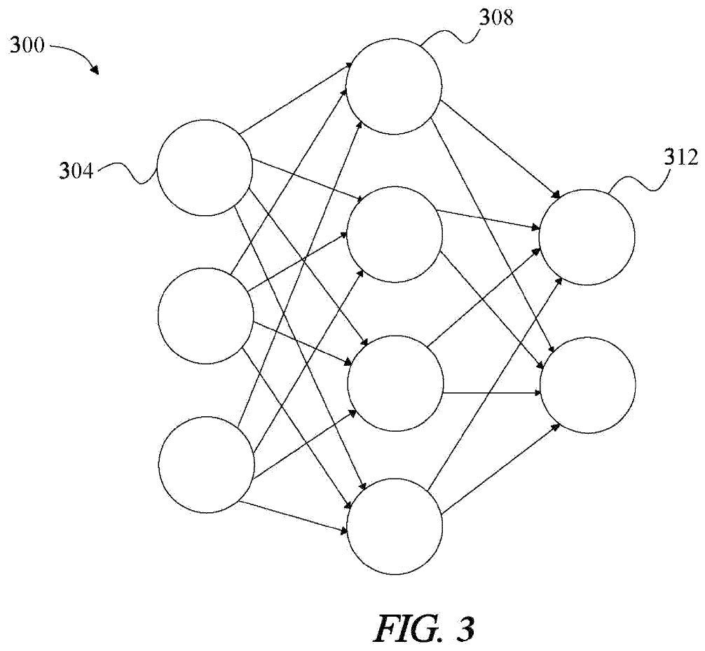

Referring now to FIG. 3, an exemplary embodiment of neural network 300 is illustrated. A neural network 300 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 304, one or more intermediate layers 308, and an output layer of nodes 312. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

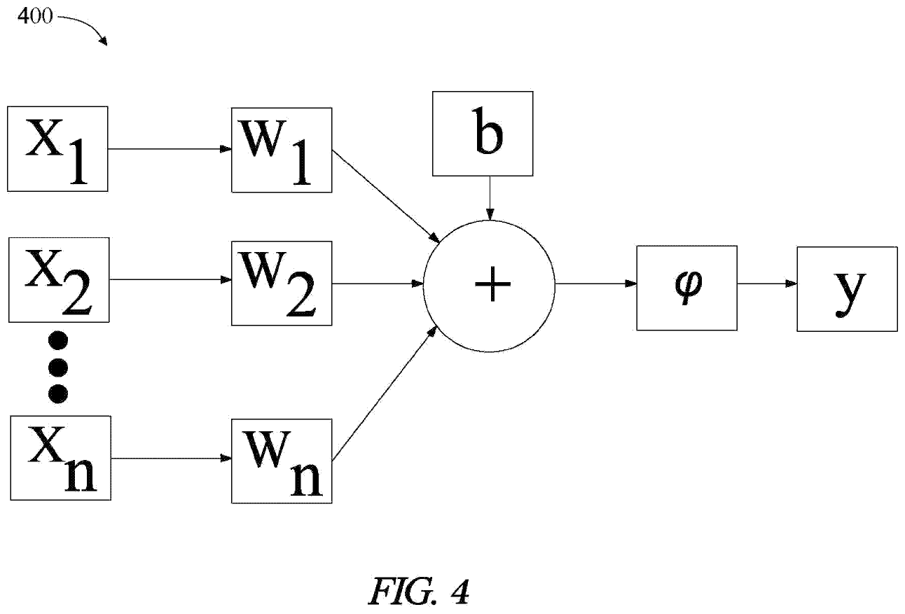

Referring now to FIG. 4, an exemplary embodiment of a node of a neural network is illustrated. A node may include, without limitation, a plurality of inputs xi that may receive numerical values from inputs to a neural network containing the node and/or from other nodes. Node may perform a weighted sum of inputs using weights wi that are multiplied by respective inputs xi. Additionally or alternatively, a bias b may be added to the weighted sum of the inputs such that an offset is added to each unit in the neural network layer that is independent of the input to the layer. The weighted sum may then be input into a function o, which may generate one or more outputs y. Weight wi applied to an input xi may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight.

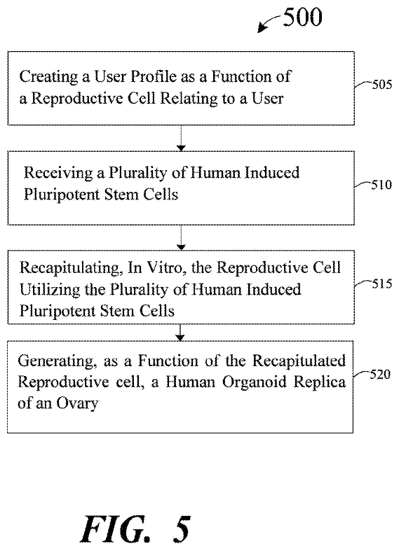

Referring now to FIG. 5, is an exemplary flow diagram of a method for engineering a human organoid replica for reproductive screening. At step 505, method 500 includes creating a user profile as a function of a reproductive cell relating to a user. In some embodiments, creating the user profile includes profiling an ovary at a single cell resolution. This may include receiving an ovarian cell from the user. “Single cell resolution,” as used in this disclosure, is next-generation sequencing technologies used to examine he sequence information from individual cells, providing a higher resolution of cellular differences and a better understanding of the function of an individual cell in the context of its microenvironment. Profiling the ovary may include utilizing in silico target discovery. In some embodiments, creating the user profile may include identifying at least an ovarian cell from an ovary demonstrating a reproductive discrepancy. A “reproductive discrepancy,” as used in this disclosure, is the abnormal cell health or function of a cell in a living organism. For example, a reproductive discrepancy may be epithelial cells dividing uncontrollably and showing signs of tumor growth. The reproductive discrepancy may include a cell health change, metabolic change, expression change, genome change, disease (e.g., endometriosis, polycystic ovary syndrome, and the like) and the like. Reproductive discrepancies may be identified using assays and machine-learning, as described above with reference to FIG. 1. For example, profiling may include high resolution profiling of transcriptome and epigenome at single cell level of ovaries from various aged cadaver donors. Profiling may include analyzing a plurality of ovaries and/or reproductive systems (e.g., fallopian tubes) received from donors of various ages to create a library of epigenetic characteristics. Donor tissue may be analyzed using methods such as oocytes for fertile co-culture, transcriptomics, metabolomics, tissue microscopy and the like. In some embodiments, donor tissue may be compared to the user profile to identity a reproductive discrepancy related to the user. For example, DNA methylation patterns exhibited in a user profile may be compared to donor tissue to compare/contrast and identity age-related diseases such as cancer, osteoarthritis, and neurodegeneration. In some embodiments, comparing a user profile to donor tissue may include utilizing a classifier as described above with reference to FIGS. 1 and 2. For example, the classifier may receive analytical data of an assayed reproductive cell relating to the user, wherein the classifier is configured to output user profile contains at least an identified reproductive discrepancy. The classifier may be trained using training data including correlation between donor tissue, a reproductive knowledge data structure containing information on reproductive health such as disease states, and any other form of data described throughout this disclosure.

At step 510, method 500 includes receiving a plurality of human induced pluripotent stem cells (hiPSCs). hiPSCs may be received from the user and/or donors. At step 515, method 500 includes recapitulating, in vitro, the ovarian cell utilizing the plurality of human induced pluripotent stem cells. In some embodiments, recapitulating the ovarian cell may include replicating an ovarian cell demonstrating a reproductive discrepancy. Recapitulating the ovarian cell may include producing engineered reproductive cells as described above with reference to FIG. 1. Recapitulating the ovarian cell further may further include utilizing transcription factor-directed cell differentiation as described above with reference to FIG. 1.

At step 520, method 500 includes generating, as a function of the recapitulated ovarian cell, a human organoid replica of an ovary as described and with reference to FIG. 1. Generating the human organoid replica may include generating a human organoid replica containing a reproductive cell, such as an ovarian cell, demonstrating a reproductive discrepancy. In some embodiments, generating the human organoid replica may include utilizing bioprinting.

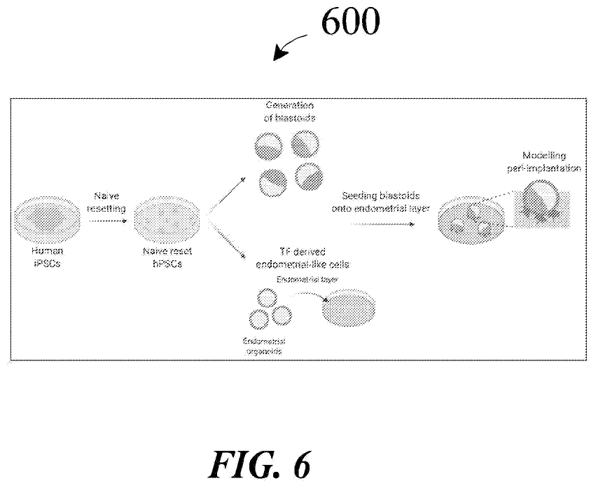

Referring now to FIG. 6, is an exemplary diagram of a human organoid replica applied as a disease model. The human organoid replica may be used in developing tools for improvement in embryo culture and implantation. During implantation, a symphony of interaction between the trophoblast originated from the trophectoderm of the implanting blastocyst and the endometrium may lead to a successful pregnancy. Defective interaction between the trophoblast and endometrium may result in implantation failure, pregnancy loss, and a number of pregnancy complications. In forming the disease model, Human iPSCs may be cultured in vitro. In some embodiments, Human iPSCs may be reprogrammed to naïve-like state. “Naïve human pluripotent stem cells (hPSC),” as used in this disclosure, are cells in vitro resembling the inner cell mass of human embryonic day (E) 6-7 preimplantation blastocysts. Compared to a primed state, cells in the naive pluripotent state may be more amenable to genome editing, present higher proliferative rate, and have higher chimeric integration potential. Additionally, naïve human pluripotent stem cells may resemble the embryonic epiblast at an earlier time-point in development than conventional, ‘primed’ hPSC. By exposing naive human embryonic stem cells to a cellular differentiation media, they may differentiate into the embryonic and the extraembryonic cell lineages, including the SOX2 positive epiblast-like cells, GATA6 positive hypoblast-like cells and GATA3 positive trophoblast-like cells. Human blastoids may be generated be generated from human embryonic stem cells. A “blastoid,” as used in this disclosure, is a stem cell-based embryo model which, morphologically and transcriptionally resembles the early, pre-implantation, mammalian conceptus, called the blastocyst.

Still referring to FIG. 6, Naïve human pluripotent stem cells may be used to generate transcription factor derived endometrial-like cells of an endometrial layer (i.e., endometrial organoids) using methods as described above with reference to at least FIG. 1. The endometrium, as used herein, is the innermost lining layer of the uterus, and functions to prevent adhesions between the opposed walls of the myometrium, thereby maintaining the patency of the uterine cavity. During the menstrual cycle or estrous cycle, the endometrium grows to a thick, blood vessel-rich, glandular tissue layer. This represents an optimal environment for the implantation of a blastocyst upon its arrival in the uterus. “Implantation,” as used in this disclosure, is the process that leads from blastocyst attachment to its embedding in the uterine wall. Failure of implantation may be linked to pregnancy loss. Toxic agents can interfere directly with the process of implantation and therefore may account for unexplained implantation failures. It may be difficult to gain a better understanding of the events in human pregnancy that occur during and just after implantation because such pregnancies may not yet be clinically detectable. Animal models of human placentation may be inadequate. In vitro models that utilize immortalized cell lines and cells derived from trophoblast cancers have multiple limitations. Primary cell and tissue cultures often have limited lifespans and cannot be obtained from the peri-implantation period. The human blastoids derived as described above may be seeded on the blastoids onto the endometrial organoid for peri-implantation modeling. Additionally, this may be used for disease modeling to discover toxins and other agents that contribute to pregnancy loss and implantation failure.

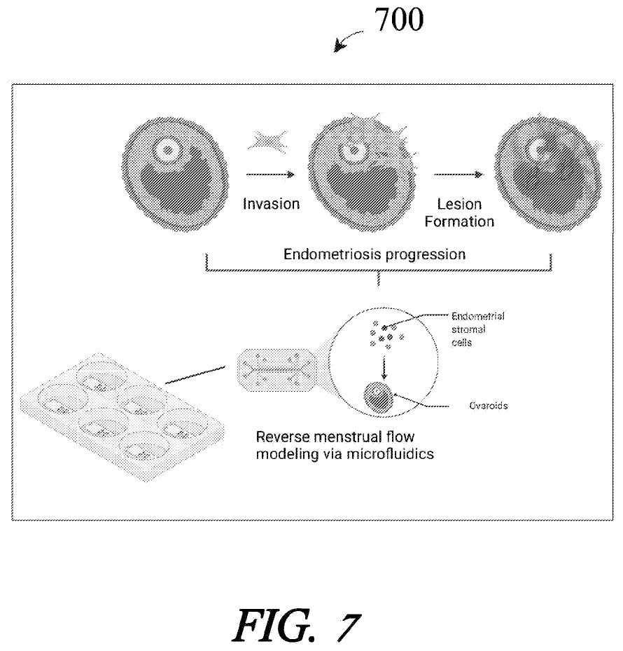

Referring now to FIG. 7, is an exemplary diagram of a human organoid replica applied as a druggable disease model. Human organoid replicas may be generated to model human endometriosis. “Endometriosis,” as used in this disclosure, is a disease of the female reproductive system in which cells similar to those in the endometrium, the layer of tissue that normally covers the inside of the uterus, grow outside the uterus. Most often this is on the ovaries, fallopian tubes, and tissue around the uterus and ovaries; in rare cases it may also occur in other parts of the body. The cause is not entirely clear. Risk factors may include having a family history of the condition. The areas of endometriosis bleed each month (menstrual period), resulting in inflammation and scarring. In some embodiments, human organoid replicas may be generated to model various stages of endometriosis progression such as invasion and lesion formation. Endometrial stromal cells may be received from a donor and cultured in vitro to derive an ovaroid (ovarian organoid). “Endometrial stromal cells,” as used in this disclosure, are the main supportive (stromal) cell type that underlies endometrial surface epithelium and surrounds glands. In the human endometrium, stromal cells may mediate the proliferative response of epithelial cells to the steroid hormones'estrogen and progesterone.

Still referring to FIG. 7, human organoid replica may be configured to model retrograde menstruation utilizing microfluidics as described above. “Retrograde menstruation,” as used in this disclosure, is the inverse flow of menstrual fluid which leaves the uterus through the fallopian tubes into the pelvic cavity. The menstrual fluid present in pelvic cavity because of the retrograde menstruation may contain the blood cells (erythrocytes) and endometrial tissue. The role of blood cells present in pelvic cavity can be considered as a complementary factor for development of endometriosis alongside the endometrial cells. Specifically, increased degradation of blood cells and insufficient inactivation of hemoglobin in pelvic cavity may be a crucial factor for the disease development. In an embodiment, human organoid replica may be configured to model any disease state and/or medical condition.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

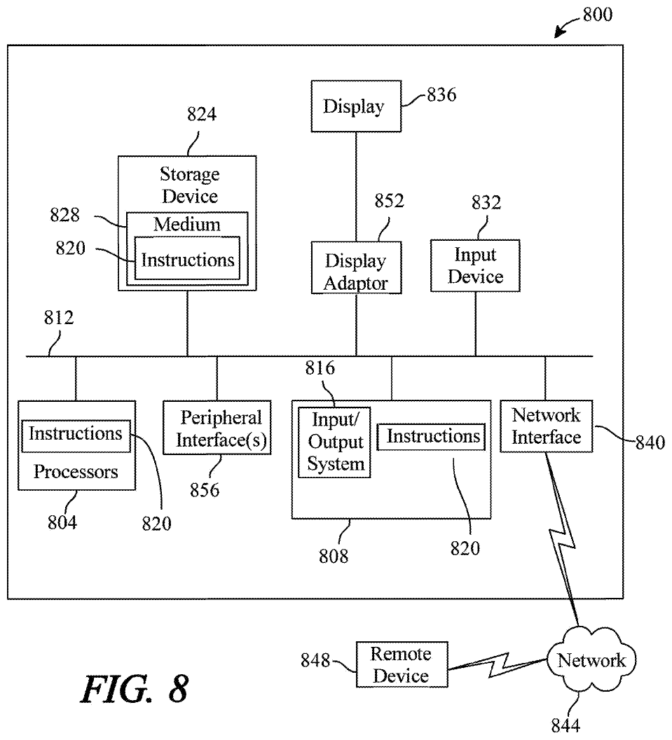

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

### EXAMPLES

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

### Example One

Producing an Ovaroid Model by Ex Vivo Differentiation of a Population of iPSCs

Using the compositions and methods described herein, one of skill in the art can produce an ovarian organoid (“ovaroid”) by differentiation, ex vivo, of a population of induced pluripotent stem cells (iPSCs, such as human iPSCs (hiPSCs)). For example, to produce an ovaroid of the disclosure, a population of hiPSCs may be transformed with one or more plasmids encoding one or more transcription factors. hiPSCs may be transformed via electroporation, liposome-mediated transformation, and viral-mediated gene transfer, among other cell transformation methodologies described herein.

In some embodiments, gene expression of a set of transcription factors of interest may be induced in a doxycycline-dependent manner. For example, to produce an ovaroid containing one or more ovarian stroma cells, one may transfect a population of hiPSCs to express one or more of transcription factors GATA4 and FOXL2. Additionally or alternatively, to produce an ovaroid containing one or more ovarian granulosa cells, one may transfect a population of hiPSCs to express one or more of transcription factors FOXL2, NR5A1, GATA4, RUNX1, and RUNX2. To obtain ovaroids that contain one or more ovarian lutein cells, one may transfect the hiPSCs so as to express one or more of transcription factors KRT19, CYP19A1, STAR, CYP17A1, and PGR. Similarly, to obtain ovaroids that contain one or more ovarian theca cells, one may transfect the hiPSCs so as to express one or more of transcription factors NR2F2 and GATA4.

Additionally or alternatively, to produce an ovaroid containing one or more human primordial germ cell-like cells (hPGCLCs), one may transfect a population of hiPSCs to express one or more of biomarkers NANOS3, CD38, ITGA6, EpCAM, BLIMP1, TFAP2C, and SOX17. One may induce differentiation of hPGCLCs from hiPSCs by supplementing growth factors, cytokines, transcription factors, or other additives to the cell medium such as, e.g., BMP4, epidermal grown factor (EGF), stem cell factor (SCF), or a combination thereof among other additives known in the art. Similarly, to produce an ovaroid containing one or more oogonia, one may transfect a population of hiPSCs to express one or more of biomarkers DDX4, DAZL, and STRA8. To produce an ovaroid containing one or more oocytes, one may transfect a population of hiPSCs to express one or more of biomarkers SYCP1, ZP1, ZP2, REC8, LHX8, and SOHLH1.

Expression of the transcription factors or other protein biomarkers described above can be evaluated by way of flow cytometry techniques, such as those known in the art and described herein.

### Example Two

### Using an Ovaroid or Uteroid Model of the Disclosure to Screen for Therapeutic Interventions Effective in Treating Diseases of the Human Female Reproductive System

Using the organoid models of the disclosure, one of skill in the art can screen candidate pharmaceutical interventions for those that are efficacious in treating a disease or condition of the human female reproductive system. For example, one may conduct such a screen by contacting a candidate pharmaceutical intervention with an organoid (e.g., an ovaroid or uteroid described herein); determining whether the organoid exhibits (i) an increase in one or more metrics of ovarian and/or uterine function relative to a measurement of the one or more metrics of ovarian and/or uterine function obtained prior to the contacting, and/or (ii) a decrease in one or more metrics of severity of the disease or condition relative to a measurement of the one or more metrics of severity of the disease or condition obtained prior to the contacting; and releasing the candidate pharmaceutical intervention for treatment of the disease or condition if the foregoing increase or decrease is observed.

The increase in the metric(s) of ovarian and/or uterine function may be an increase of, e.g., 1%, 2%, 3%, 4%, 5% ,10%, 20%, 30%, 40%, 50%, 60%, 70%, 80%, 90%, 100%, 200%, 500%, or more, relative to a measurement of the metric(s) of ovarian and/or uterine function obtained prior to the contacting step. Similarly, the decrease in the metric(s) of severity of the disease or condition may be a decrease of, e.g., 1%, 2%, 3%, 4%, 5% ,10%, 20%, 30%, 40%, 50%, 60%, 70%, 80%, 90%, 95%, or more, relative to a measurement of the metric(s) of severity of the disease or condition obtained prior to the contacting step.

The one or more metrics of ovarian and/or uterine function may include, e.g., the rate and/or extent to which the organoid matures an immature oocyte upon co-culturing the organoid with the immature oocyte; the rate and/or extent to which the organoid secretes an estrogen or progestogen; the viability of the organoid or a component cell type thereof; and/or the rate and/or extent to which the organoid specifically binds to one or more cells that model an embryo.

Additionally or alternatively, the one or more metrics of severity of the disease or condition may include, e.g., abnormal or aberrant growth of the organoid or a component cell type thereof; the extent to which the organoid secretes an estrogen or progestogen above or below a reference level of estrogen or progestogen secretion; and/or the rate at which the organoid or a component cell type thereof undergoes necrosis, apoptosis, or other form of cell death.

Using screens of this nature, one may assess the efficacy of a therapeutic intervention for the treatment of a variety of diseases or conditions. For example, the disease or condition may be one that is associated with a decline in ovarian function. In some embodiments, the condition is a timewise reduction in ovarian function. In some embodiments, the timewise reduction in ovarian function is due to menopause, such as premature menopause. In some embodiments, the decline in ovarian function is a decline in one or more of follicular development, oocyte release, and oocyte maturation.

Exemplary diseases for which a therapeutic intervention may be screened using the methodology described above include, without limitation, primary ovarian insufficiency (POI), polycystic ovarian syndrome (PCOS), ovarian cancer, or ovarian hyperstimulation syndrome. In some embodiments, the disease or condition is one that adversely affects uterine cell function or viability. In some embodiments, the disease or condition is endometriosis, uterine fibroids, adenomyosis, a gynecological cancer, pelvic inflammatory disease (PID), cervical dysplasia, or pelvic floor prolapse. In some embodiments, the disease or condition is one that reduces the subject's fertility. In some embodiments, the disease or condition is embryo implantation failure.

### Example Three

Using an Ovaroid or Uteroid Model of the Disclosure to Conduct Toxicology screens

Using the organoid models of the disclosure, one of skill in the art can screen candidate pharmaceutical interventions to evaluate their safety for administration to a female subject (e.g., a human female subject). For example, to conduct such a screen, one may use the compositions and methods described herein to contact a candidate pharmaceutical intervention with an organoid (e.g., an ovaroid or uteroid model) and determine whether the organoid exhibits (i) a decrease in one or more metrics of ovarian and/or uterine function relative to a measurement of the one or more metrics of ovarian and/or uterine function obtained prior to the contacting, and/or (ii) an increase in one or more indicators of a gynecological disorder relative to a measurement of the one or more metrics of a gynecological disorder obtained prior to the contacting. If the organoid does not exhibit a decrease in one or more of the above metrics of ovarian and/or uterine function, and/or if the organoid does not exhibit an increase in one or more of the above indicators of a gynecological disorder, then the candidate pharmaceutical intervention may be released for administration to a female subject.

Using the compositions and methods of the disclosure, one can evaluate the safety of a variety of therapeutic modalities. For example, the candidate pharmaceutical intervention may be a small molecule, a polynucleotide (e.g., an antisense oligonucleotide, siRNA, shRNA, or miRNA), a polypeptide (e.g., an antibody or antigen-binding fragment thereof), a gene therapy (e.g., a DNA or RNA vector, optionally wherein the vector is a viral vector), or a cell therapy (e.g., a chimeric antigen receptor T-cell (CAR-T cell) therapy).

A variety of metrics of ovarian and/or uterine function may be used to assess safety in the foregoing screens. For example, metrics of ovarian and/or uterine function may include the rate and/or extent to which the organoid matures an immature oocyte upon co-culturing the organoid with the immature oocyte; the rate and/or extent to which the organoid secretes an estrogen or progestogen; the viability of the organoid or a component cell type thereof; and/or the rate and/or extent to which the organoid specifically binds to one or more cells that model an embryo.

Similarly, a variety of indicators of a gynecological disorder may be used to assess safety. These metrics include, without limitation, abnormal or aberrant growth of the organoid or a component cell type thereof; the extent to which the organoid secretes an estrogen or progestogen above or below a reference level of estrogen or progestogen secretion; and/or the rate at which the organoid or a component cell type thereof undergoes necrosis, apoptosis, or other form of cell death.

In exemplary embodiments, the reference level is a level of estrogen (e.g., estradiol) secretion that is ordinarily observed in a healthy, pre-menopausal human female subject that does not have the disease or condition. In some embodiments, the reference level of estradiol secretion is a concentration that is within the “Barbieri window” of healthy estradiol secretion, e.g., within a range of from about 20 pg/ml to about 50 pg/ml (e.g., 20 pg/ml, 21 pg/ml, 22 pg/ml, 23 pg/ml, 24 pg/ml, 25 pg/ml, 26 pg/ml, 27 pg/ml, 28 pg/ml, 29 pg/ml, 30 pg/ml, 31 pg/ml, 32 pg/ml, 33 pg/ml, 34 pg/ml, 35 pg/ml, 36 pg/ml, 37 pg/ml, 38 pg/ml, 39 pg/ml, 40 pg/ml, 41 pg/ml, 42 pg/ml, 43 pg/ml, 44 pg/ml, 45 pg/ml, 46 pg/ml, 47 pg/ml, 48 pg/ml, 49 pg/ml, or 50 pg/ml).

If, using the foregoing screen, a candidate pharmaceutical intervention is determined to be safe for administration to a female subject, the intervention may be released either for administration to a patient (e.g., a patient having a pathology described herein) or may progress to further testing (e.g., to testing for therapeutic efficacy). In this way, the efficacy screening paradigm (Example Two) and safety screening paradigm (Example Three) described herein may be used synergistically: those interventions that are identified as being safe in one assay may then be assessed for efficacy in another, and vice versa.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations following, in general, the principles and including such departures from the invention that come within known or customary practice within the art to which the invention pertains and may be applied to the essential features hereinbefore set forth, and follows in the scope of the claims.

Other embodiments are within the claims.

1. An organoid comprising a population of cells of a human female reproductive organ and an extracellular matrix (ECM) substrate, wherein the cells are obtained by differentiation, ex vivo, of one or more pluripotent stem or progenitor cells, and wherein the population of cells comprises:
a) a plurality of ovarian granulosa cells, ovarian stroma cells, ovarian lutein cells, ovarian theca cells, and/or germ cells; and/or
b) a plurality of uterine endometrial cells, uterine myometrial cells, and/or uterine perimetrial cells.

2. The organoid of claim 1, wherein the organ is an ovary and the population of cells comprises a plurality of ovarian granulosa cells, ovarian stroma cells, ovarian lutein cells, ovarian theca cells and/or germ cells.

3. The organoid of claim 1 or 2, wherein the population of cells comprises a plurality of ovarian granulosa cells.

4. The organoid of any one of claims 1-3, wherein the ovarian granulosa cells express one or more, or all, of proteins CD82, follicle-stimulating hormone receptor (FSHR), FOXL2, NR5A1, GATA4, RUNX1, and RUNX2.

5. The organoid of any one of claims 1-4, wherein the ovarian granulosa cells secrete an estrogen.

6. The organoid of claim 5, wherein the estrogen is estradiol.

7. The organoid of any one of claims 1-6, wherein the population of cells comprises from about 1×10⁵ to about 1×10⁸ ovarian granulosa cells.

8. The organoid of claim 7, wherein the population of cells comprises from about 1×10⁵ to about 2×10⁵ ovarian granulosa cells, from about 2×10⁵ to about 3×10⁵ ovarian granulosa cells, from about 3×10⁵ to about 4×10⁵ ovarian granulosa cells, from about 4×10⁵ to about 5×10⁵ ovarian granulosa cells, from about 5×10⁵ to about 6×10⁵ ovarian granulosa cells, from about 6×10⁵ to about 7×10⁵ ovarian granulosa cells, from about 7×10⁵ to about 8×10⁵ ovarian granulosa cells, from about 8×10⁵ to about 9×10⁵ ovarian granulosa cells, or from about 9×10⁵ to about 1×10⁶ ovarian granulosa cells.

9. The organoid of claim 7, wherein the population of cells comprises from about 1×10⁶ to about 2×10⁶ ovarian granulosa cells, from about 2×10⁶ to about 3×10⁶ ovarian granulosa cells, from about 3×10⁶ to about 4×10⁶ ovarian granulosa cells, from about 4×10⁶ to about 5×10⁶ ovarian granulosa cells, from about 5×10⁶ to about 6×10⁶ ovarian granulosa cells, from about 6×10⁶ to about 7×10⁶ ovarian granulosa cells, from about 7×10⁶ to about 8×10⁶ ovarian granulosa cells, from about 8×10⁶ to about 9×10⁶ ovarian granulosa cells, or from about 9×10⁶ to about 1×10⁷ ovarian granulosa cells.

10. The organoid of claim 7, wherein the population of cells comprises from about 1×10⁷ to about 2×10⁷ ovarian granulosa cells, from about 2×10⁷ to about 3×10⁷ ovarian granulosa cells, from about 3×10⁷ to about 4×10⁷ ovarian granulosa cells, from about 4×10⁷ to about 5×10⁷ ovarian granulosa cells, from about 5×10⁷ to about 6×10⁷ ovarian granulosa cells, from about 6×10⁷ to about 7×10⁷ ovarian granulosa cells, from about 7×10⁷ to about 8×10⁷ ovarian granulosa cells, from about 8×10⁷ to about 9×10⁷ ovarian granulosa cells, or from about 9×10⁷ to about 1×10⁸ ovarian granulosa cells.

11. The organoid of claim 7, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian granulosa cells.

12. The organoid of any one of claims 1-11, wherein the population of cells comprises a plurality of ovarian stroma cells.

13. The organoid of any one of claims 1-12, wherein the ovarian stroma cells express NR2F2.

14. The organoid of any one of claims 1-13, wherein the population of cells comprises from about 1×10⁵ to about 1×10⁸ ovarian stroma cells.

15. The organoid of claim 14, wherein the population of cells comprises from about 1×10⁵ to about 2×10⁵ ovarian stroma cells, from about 2×10⁵ to about 3×10⁵ ovarian stroma cells, from about 3×10⁵ to about 4×10⁵ ovarian stroma cells, from about 4×10⁵ to about 5×10⁵ ovarian stroma cells, from about 5×10⁵ to about 6×10⁵ ovarian stroma cells, from about 6×10⁵ to about 7×10⁵ ovarian stroma cells, from about 7×10⁵ to about 8×10⁵ ovarian stroma cells, from about 8×10⁵ to about 9×10⁵ ovarian stroma cells, or from about 9×10⁵ to about 1×10⁶ ovarian stroma cells.

16. The organoid of claim 14, wherein the population of cells comprises from about 1×10⁶ to about 2×10⁶ ovarian stroma cells, from about 2×10⁶ to about 3×10⁶ ovarian stroma cells, from about 3×10⁶ to about 4×10⁶ ovarian stroma cells, from about 4×10⁶ to about 5×10⁶ ovarian stroma cells, from about 5×10⁶ to about 6×10⁶ ovarian stroma cells, from about 6×10⁶ to about 7×10⁶ ovarian stroma cells, from about 7×10⁶ to about 8×10⁶ ovarian stroma cells, from about 8×10⁶ to about 9×10⁶ ovarian stroma cells, or from about 9×10⁶ to about 1×10⁷ ovarian stroma cells.

17. The organoid of claim 14, wherein the population of cells comprises from about 1×10⁷ to about 2×10⁷ ovarian stroma cells, from about 2×10⁷ to about 3×10⁷ ovarian stroma cells, from about 3×10⁷ to about 4×10⁷ ovarian stroma cells, from about 4×10⁷ to about 5×10⁷ ovarian stroma cells, from about 5×10⁷ to about 6×10⁷ ovarian stroma cells, from about 6×10⁷ to about 7×10⁷ ovarian stroma cells, from about 7×10⁷ to about 8×10⁷ ovarian stroma cells, from about 8×10⁷ to about 9×10⁷ ovarian stroma cells, or from about 9×10⁷ to about 1×10⁸ ovarian stroma cells.

18. The organoid of claim 14, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian stroma cells.

19. The organoid of any one of claims 1-18, wherein the population of cells comprises a plurality of ovarian lutein cells.

20. The organoid of any one of claims 1-19, wherein the ovarian lutein cells express one or more, or all, of proteins KRT19, CYP19A1, STAR, CYP17A1, and PGR.

21. The organoid of any one of claims 1-20, wherein the ovarian lutein cells secrete a progestogen.

22. The organoid of claim 21, wherein the progestogen is progesterone.

23. The organoid of any one of claims 1-22, wherein the population of cells comprises from about 1×10⁵ to about 1×10⁸ ovarian lutein cells.

24. The organoid of claim 23, wherein the population of cells comprises from about 1×10⁵ to about 2×10⁵ ovarian lutein cells, from about 2×10⁵ to about 3×10⁵ ovarian lutein cells, from about 3×10⁵ to about 4×10⁵ ovarian lutein cells, from about 4×10⁵ to about 5×10⁵ ovarian lutein cells, from about 5×10⁵ to about 6×10⁵ ovarian lutein cells, from about 6×10⁵ to about 7×10⁵ ovarian lutein cells, from about 7×10⁵ to about 8×10⁵ ovarian lutein cells, from about 8×10⁵ to about 9×10⁵ ovarian lutein cells, or from about 9×10⁵ to about 1×10⁶ ovarian lutein cells.

25. The organoid of claim 23, wherein the population of cells comprises from about 1×10⁶ to about 2×10⁶ ovarian lutein cells, from about 2×10⁶ to about 3×10⁶ ovarian lutein cells, from about 3×10⁶ to about 4×10⁶ ovarian lutein cells, from about 4×10⁶ to about 5×10⁶ ovarian lutein cells, from about 5×10⁶ to about 6×10⁶ ovarian lutein cells, from about 6×10⁶ to about 7×10⁶ ovarian lutein cells, from about 7×10⁶ to about 8×10⁶ ovarian lutein cells, from about 8×10⁶ to about 9×10⁶ ovarian lutein cells, or from about 9×10⁶ to about 1×10⁷ ovarian lutein cells.

26. The organoid of claim 23, wherein the population of cells comprises from about 1×10⁷ to about 2×10⁷ ovarian lutein cells, from about 2×10⁷ to about 3×10⁷ ovarian lutein cells, from about 3×10⁷ to about 4×10⁷ ovarian lutein cells, from about 4×10⁷ to about 5×10⁷ ovarian lutein cells, from about 5×10⁷ to about 6×10⁷ ovarian lutein cells, from about 6×10⁷ to about 7×10⁷ ovarian lutein cells, from about 7×10⁷ to about 8×10⁷ ovarian lutein cells, from about 8×10⁷ to about 9×10⁷ ovarian lutein cells, or from about 9×10⁷ to about 1×10⁸ ovarian lutein cells.

27. The organoid of claim 23, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian lutein cells.

28. The organoid of any one of claims 1-27, wherein the population of cells comprises a plurality of ovarian theca cells.

29. The organoid of any one of claims 1-28, wherein the ovarian theca cells express one or both of proteins NR2F2 and GATA4.

30. The organoid of any one of claims 1-29, wherein the ovarian theca cells secrete an androgen.

31. The organoid of claim 30, wherein the androgen is androstenedione.

32. The organoid of any one of claims 1-31, wherein the population of cells comprises from about 1×10⁵ to about 1×10⁸ ovarian theca cells.

33. The organoid of claim 32, wherein the population of cells comprises from about 1×10⁵ to about 2×10⁵ ovarian theca cells, from about 2×10⁵ to about 3×10⁵ ovarian theca cells, from about 3×10⁵ to about 4×10⁵ ovarian theca cells, from about 4×10⁵ to about 5×10⁵ ovarian theca cells, from about 5×10⁵ to about 6×10⁵ ovarian theca cells, from about 6×10⁵ to about 7×10⁵ ovarian theca cells, from about 7×10⁵ to about 8×10⁵ ovarian theca cells, from about 8×10⁵ to about 9×10⁵ ovarian theca cells, or from about 9×10⁵ to about 1×10⁶ ovarian theca cells.

34. The organoid of claim 32, wherein the population of cells comprises from about 1×10⁶ to about 2×10⁶ ovarian theca cells, from about 2×10⁶ to about 3×10⁶ ovarian theca cells, from about 3×10⁶ to about 4×10⁶ ovarian theca cells, from about 4×10⁶ to about 5×10⁶ ovarian theca cells, from about 5×10⁶ to about 6×10⁶ ovarian theca cells, from about 6×10⁶ to about 7×10⁶ ovarian theca cells, from about 7×10⁶ to about 8×10⁶ ovarian theca cells, from about 8×10⁶ to about 9×10⁶ ovarian theca cells, or from about 9×10⁶ to about 1×10⁷ ovarian theca cells.

35. The organoid of claim 32, wherein the population of cells comprises from about 1×10⁷ to about 2×10⁷ ovarian theca cells, from about 2×10⁷ to about 3×10⁷ ovarian theca cells, from about 3×10⁷ to about 4×10⁷ ovarian theca cells, from about 4×10⁷ to about 5×10⁷ ovarian theca cells, from about 5×10⁷ to about 6×10⁷ ovarian theca cells, from about 6×10⁷ to about 7×10⁷ ovarian theca cells, from about 7×10⁷ to about 8×10⁷ ovarian theca cells, from about 8×10⁷ to about 9×10⁷ ovarian theca cells, or from about 9×10⁷ to about 1×10⁸ ovarian theca cells.

36. The organoid of claim 32, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ ovarian theca cells.

37. The organoid of any one of claims 1-36, wherein the germ cells comprise one or more human primordial germ cell-like cells (hPGCLCs), oogonia, and/or oocytes, optionally wherein the germ cells comprise one or more hPGCLCs, oogonia, and oocytes.

38. The organoid of any one of claims 1-37, wherein the germ cells comprise one or more

39. The organoid of any one of claims 1-38, wherein the hPGCLCs express one or more, or all, of proteins NANOS3, CD38, ITGA6, EpCAM, BLIMP1, TFAP2C and SOX17.

40. The organoid of any one of claims 1-39, wherein the population of cells comprises from about 1×10⁵ to about 1×10⁸ hPGCLCs.

41. The organoid of claim 40, wherein the population of cells comprises from about 1×10⁵ to about 2×10⁵ hPGCLCs, from about 2×10⁵ to about 3×10⁵ hPGCLCs, from about 3×10⁵ to about 4×10⁵ hPGCLCs, from about 4×10⁵ to about 5×10⁵ hPGCLCs, from about 5×10⁵ to about 6×10⁵ hPGCLCs, from about 6×10⁵ to about 7×10⁵ hPGCLCs, from about 7×10⁵ to about 8×10⁵ hPGCLCs, from about 8×10⁵ to about 9×10⁵ hPGCLCs, or from about 9×10⁵ to about 1×10⁶ hPGCLCs.

42. The organoid of claim 40, wherein the population of cells comprises from about 1×10⁶ to about 2×10⁶ hPGCLCs, from about 2×10⁶ to about 3×10⁶ hPGCLCs, from about 3×10⁶ to about 4×10⁶ hPGCLCs, from about 4×10⁶ to about 5×10⁶ hPGCLCs, from about 5×10⁶ to about 6×10⁶ hPGCLCs, from about 6×10⁶ to about 7×10⁶ hPGCLCs, from about 7×10⁶ to about 8×10⁶ hPGCLCs, from about 8×10⁶ to about 9×10⁶ hPGCLCs, or from about 9×10⁶ to about 1×10⁷ hPGCLCs.

43. The organoid of claim 40, wherein the population of cells comprises from about 1×10⁷ to about 2×10⁷ hPGCLCs, from about 2×10⁷ to about 3×10⁷ hPGCLCs, from about 3×10⁷ to about 4×10⁷ hPGCLCs, from about 4×10⁷ to about 5×10⁷ hPGCLCs, from about 5×10⁷ to about 6×10⁷ hPGCLCs, from about 6×10⁷ to about 7×10⁷ hPGCLCs, from about 7×10⁷ to about 8×10⁷ hPGCLCs, from about 8×10⁷ to about 9×10⁷ hPGCLCs, or from about 9×10⁷ to about 1×10⁸ hPGCLCs.

44. The organoid of claim 40, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ hPGCLCs.

45. The organoid of any one of claims 1-44, wherein the germ cells comprise one or more oogonia.

46. The organoid of any one of claims 1-45, wherein the oogonia express one or more, or all, of proteins DDX4, DAZL, and STRA8.

47. The organoid of any one of claims 1-46, wherein the population of cells comprises from about 1×10⁵ to about 1×10⁸ oogonia.

48. The organoid of claim 47, wherein the population of cells comprises from about 1×10⁵ to about 2×10⁵ oogonia, from about 2×10⁵ to about 3×10⁵ oogonia, from about 3×10⁵ to about 4×10⁵ oogonia, from about 4×10⁵ to about 5×10⁵ oogonia, from about 5×10⁵ to about 6×10⁵ oogonia, from about 6×10⁵ to about 7×10⁵ oogonia, from about 7×10⁵ to about 8×10⁵ oogonia, from about 8×10⁵ to about 9×10⁵ oogonia, or from about 9×10⁵ to about 1×10⁶ oogonia.

49. The organoid of claim 47, wherein the population of cells comprises from about 1×10⁶ to about 2×10⁶ oogonia, from about 2×10⁶ to about 3×10⁶ oogonia, from about 3×10⁶ to about 4×10⁶ oogonia, from about 4×10⁶ to about 5×10⁶ oogonia, from about 5×10⁶ to about 6×10⁶ oogonia, from about 6×10⁶ to about 7×10⁶ oogonia, from about 7×10⁶ to about 8×10⁶ oogonia, from about 8×10⁶ to about 9×10⁶ oogonia, or from about 9×10⁶ to about 1×10⁷ oogonia.

50. The organoid of claim 47, wherein the population of cells comprises from about 1×10⁷ to about 2×10⁷ oogonia, from about 2×10⁷ to about 3×10⁷ oogonia, from about 3×10⁷ to about 4×10⁷ oogonia, from about 4×10⁷ to about 5×10⁷ oogonia, from about 5×10⁷ to about 6×10⁷ oogonia, from about 6×10⁷ to about 7×10⁷ oogonia, from about 7×10⁷ to about 8×10⁷ oogonia, from about 8×10⁷ to about 9×10⁷ oogonia, or from about 9×10⁷ to about 1×10⁸ oogonia.

51. The organoid of claim 47, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ oogonia.

52. The organoid of any one of claims 1-51, wherein the germ cells comprise one or more oocytes.

53. The organoid of any one of claims 1-52, wherein the oocytes express one or more, or all, of proteins SYCP1, ZP1, ZP2, REC8, LHX8, and SOHLH1.

54. The organoid of any one of claims 1-53, wherein the population of cells comprises from about 1×10⁵ to about 1×10⁸ oocytes.

55. The organoid of claim 54, wherein the population of cells comprises from about 1×10⁵ to about 2×10⁵ oocytes, from about 2×10⁵ to about 3×10⁵ oocytes, from about 3×10⁵ to about 4×10⁵ oocytes, from about 4×10⁵ to about 5×10⁵ oocytes, from about 5×10⁵ to about 6×10⁵ oocytes, from about 6×10⁵ to about 7×10⁵ oocytes, from about 7×10⁵ to about 8×10⁵ oocytes, from about 8×10⁵ to about 9×10⁵ oocytes, or from about 9×10⁵ to about 1×10⁶ oocytes.

56. The organoid of claim 54, wherein the population of cells comprises from about 1×10⁶ to about 2×10⁶ oocytes, from about 2×10⁶ to about 3×10⁶ oocytes, from about 3×10⁶ to about 4×10⁶ oocytes, from about 4×10⁶ to about 5×10⁶ oocytes, from about 5×10⁶ to about 6×10⁶ oocytes, from about 6×10⁶ to about 7×10⁶ oocytes, from about 7×10⁶ to about 8×10⁶ oocytes, from about 8×10⁶ to about 9×10⁶ oocytes, or from about 9×10⁶ to about 1×10⁷ oocytes.

57. The organoid of claim 54, wherein the population of cells comprises from about 1×10⁷ to about 2×10⁷ oocytes, from about 2×10⁷ to about 3×10⁷ oocytes, from about 3×10⁷ to about 4×10⁷ oocytes, from about 4×10⁷ to about 5×10⁷ oocytes, from about 5×10⁷ to about 6×10⁷ oocytes, from about 6×10⁷ to about 7×10⁷ oocytes, from about 7×10⁷ to about 8×10⁷ oocytes, from about 8×10⁷ to about 9×10⁷ oocytes, or from about 9×10⁷ to about 1×10⁸ oocytes.

58. The organoid of claim 54, wherein the population of cells comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ oocytes.

59. The organoid of any one of claims 1-58, wherein the pluripotent stem or progenitor cells are induced pluripotent stem cells (iPSCs).

60. The organoid of claim 59, wherein the organ is an ovary, the population of cells comprises a plurality of ovarian granulosa cells, ovarian stroma cells, ovarian lutein cells, and/or ovarian theca cells, and the population of cells is obtained by modifying the iPSCs to express one or more, or all five, of FOXL2, NR5A1, GATA4, RUNX1, and RUNX2.

61. The organoid of any one of claims 1-60, wherein the organoid further comprises an extracellular matrix (ECM) substrate.

62. The organoid of claim 61, wherein the ECM substrate comprises one or more of collagen, an epidermal growth factor (EGF), an elastin, a fibronectin, a vitronectin, a laminin, a cell adhesion protein, or a plant-derived protein or protein polymer.

63. The organoid of claim 62, wherein the collagen is fibrillar collagen.

64. The organoid of claim 62 or 63, wherein the collagen is collagen type I, type II, type III, type V, type XI.

65. The organoid of any one of claims 62-64, wherein the elastin is tropoelastin or mature elastin.

66. The organoid of any one of claims 62-65, wherein the plant-derived protein polymer is alginate.

67. The organoid of claim 1, wherein the organ is a uterus and the population of cells comprises a plurality of uterine endometrial cells, uterine myometrial cells, and/or uterine perimetrial cells.

68. The organoid of claim 67, wherein the population of cells comprises a plurality of uterine endometrial cells, uterine myometrial cells, and uterine perimetrial cells.

69. A method of producing an organoid comprising a population of human ovarian cells, the method comprising:
a) introducing, into one or more pluripotent stem or progenitor cells, one or more nucleic acid molecules that collectively encode FOXL2, NR5A1, GATA4, RUNX1, and/or RUNX2, thereby differentiating the one or more pluripotent stem or progenitor cells into a population of ovarian granulosa cells, ovarian stroma cells, ovarian lutein cells, and/or ovarian theca cells; and
b) contacting the cells resulting from (a) with an ECM substrate, optionally wherein the ECM substrate comprises one or more of collagen, an EGF, an elastin, a fibronectin, a vitronectin, a laminin, a cell adhesion protein, or a plant-derived protein or protein polymer.

70. The method of claim 69, further comprising introducing into the one or more pluripotent stem or progenitor cells, one or more nucleic acid molecules that collectively encode NANOS3, CD38, ITGA6, EpCAM, BLIMP1, TFAP2C, and/or SOX17, thereby differentiating the one or more pluripotent stem or progenitor cells into a population of germ cells, optionally wherein the germ cells comprise one or more hPGCLCs, oogonia, and/or oocytes.

71. The method of claim 70, wherein the one or more nucleic acid molecules are introduced into the one or more pluripotent stem or progenitor cells by way of electroporation.

72. The method of claim 70 or 71, wherein the one or more nucleic acid molecules collectively encode at least two of FOXL2, NR5A1, GATA4, RUNX1, and RUNX2.

73. The method of claim 72, wherein the one or more nucleic acid molecules collectively encode at least three of FOXL2, NR5A1, GATA4, RUNX1, and RUNX2.

74. The method of claim 73, wherein the one or more nucleic acid molecules collectively encode at least four of FOXL2, NR5A1, GATA4, RUNX1, and RUNX2.

75. The method of claim 74, wherein the one or more nucleic acid molecules collectively encode all five of FOXL2, NR5A1, GATA4, RUNX1, and RUNX2.

76. The method of any one of claims 69-75, wherein the organoid comprises from about 1×10⁵ to about 1×10⁸ pluripotent stem or progenitor cells.

77. The method of claim 76, wherein the organoid comprises from about 1×10⁵ to about 2×10⁵ pluripotent stem or progenitor cells, from about 2×10⁵ to about 3×10⁵ pluripotent stem or progenitor cells, from about 3×10⁵ to about 4×10⁵ pluripotent stem or progenitor cells, from about 4×10⁵ to about 5×10⁵ pluripotent stem or progenitor cells, from about 5×10⁵ to about 6×10⁵ pluripotent stem or progenitor cells, from about 6×10⁵ to about 7×10⁵ pluripotent stem or progenitor cells, from about 7×10⁵ to about 8×10⁵ pluripotent stem or progenitor cells, from about 8×10⁵ to about 9×10⁵ pluripotent stem or progenitor cells, or from about 9×10⁵ to about 1×10⁶ pluripotent stem or progenitor cells.

78. The method of claim 76, wherein the organoid comprises from about 1×10⁶ to about 2×10⁶ pluripotent stem or progenitor cells, from about 2×10⁶ to about 3×10⁶ pluripotent stem or progenitor cells, from about 3×10⁶ to about 4×10⁶ pluripotent stem or progenitor cells, from about 4×10⁶ to about 5×10⁶ pluripotent stem or progenitor cells, from about 5×10⁶ to about 6×10⁶ pluripotent stem or progenitor cells, from about 6×10⁶ to about 7×10⁶ pluripotent stem or progenitor cells, from about 7×10⁶ to about 8×10⁶ pluripotent stem or progenitor cells, from about 8×10⁶ to about 9×10⁶ pluripotent stem or progenitor cells, or from about 9×10⁶ to about 1×10⁷ pluripotent stem or progenitor cells.

79. The method of claim 76, wherein the organoid comprises from about 1×10⁷ to about 2×10⁷ pluripotent stem or progenitor cells, from about 2×10⁷ to about 3×10⁷ pluripotent stem or progenitor cells, from about 3×10⁷ to about 4×10⁷ pluripotent stem or progenitor cells, from about 4×10⁷ to about 5×10⁷ pluripotent stem or progenitor cells, from about 5×10⁷ to about 6×10⁷ pluripotent stem or progenitor cells, from about 6×10⁷ to about 7×10⁷ pluripotent stem or progenitor cells, from about 7×10⁷ to about 8×10⁷ pluripotent stem or progenitor cells, from about 8×10⁷ to about 9×10⁷ pluripotent stem or progenitor cells, or from about 9×10⁷ to about 1×10⁸ pluripotent stem or progenitor cells.

80. The method of claim 76, wherein the organoid comprises about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸ pluripotent stem or progenitor cells.

81. The method of any one of claims 70-80, wherein the pluripotent stem or progenitor cells are iPSCs.

82. The method of any one of claims 69-81, wherein the collagen is fibrillar collagen.

83. The method of any one of claims 69-82, wherein the collagen is collagen type I, type II, type III, type V, type XI.

84. The method of any one of claims 69-83, wherein the elastin is tropoelastin or mature elastin.

85. The method of any one of claims 69-84, wherein the plant-derived protein polymer is alginate.

86. A method of determining whether a candidate pharmaceutical intervention is efficacious in treating a disease or condition of the human female reproductive system, the method comprising:
a) contacting the candidate pharmaceutical intervention with the organoid of any one of claims 1-68;
b) determining that the organoid exhibits (i) an increase in one or more metrics of ovarian and/or uterine function relative to a measurement of the one or more metrics of ovarian and/or uterine function obtained prior to the contacting, and/or (ii) a decrease in one or more metrics of severity of the disease or condition relative to a measurement of the one or more metrics of severity of the disease or condition obtained prior to the contacting; and, optionally,
c) releasing the candidate pharmaceutical intervention for treatment of the disease or condition in a subject in need thereof.

87. The method of claim 86, further comprising:
d) administering a therapeutically effective amount of the candidate pharmaceutical intervention to the subject.

88. A method of treating or preventing a disease or condition of the human female reproductive system in a subject in need thereof, the method comprising:
a) contacting the candidate pharmaceutical intervention with the organoid of any one of claims 1-68;
b) determining that the organoid exhibits (i) an increase in one or more metrics of ovarian and/or uterine function relative to a measurement of the one or more metrics of ovarian and/or uterine function obtained prior to the contacting, and/or (ii) a decrease in one or more metrics of severity of the disease or condition relative to a measurement of the one or more metrics of severity of the disease or condition obtained prior to the contacting; and
c) administering a therapeutically effective amount of the candidate pharmaceutical intervention to the subject.

89. A method of treating or preventing a disease or condition of the human female reproductive system in a subject in need thereof, the method comprising administering to the subject a therapeutically effective amount of a candidate pharmaceutical intervention, wherein the candidate pharmaceutical intervention has previously been identified as being efficacious in treating the disease or condition by a method comprising:
a) contacting the candidate pharmaceutical intervention with the organoid of any one of claims 1-68; and
b) determining that the organoid exhibits (i) an increase in one or more metrics of ovarian and/or uterine function relative to a measurement of the one or more metrics of ovarian and/or uterine function obtained prior to the contacting, and/or (ii) a decrease in one or more metrics of severity of the disease or condition relative to a measurement of the one or more metrics of severity of the disease or condition obtained prior to the contacting.

90. The method of any one of claims 86-89, wherein the subject is a human.

91. The method of any one of claims 86-90, wherein the disease or condition is one that is associated with a decline in ovarian function.

92. The method of any one of claims 86-91, wherein the condition is a timewise reduction in ovarian function.

93. The method of claim 92, wherein the timewise reduction in ovarian function is due to menopause, optionally wherein the menopause is premature menopause.

94. The method of any one of claims 91-93, wherein the decline in ovarian function is a decline in one or more of follicular development, oocyte release, and oocyte maturation.

95. The method of any one of claims 91-94, wherein the disease is primary ovarian insufficiency (POI), polycystic ovarian syndrome (PCOS), ovarian cancer, or ovarian hyperstimulation syndrome.

96. The method of any one of claims 86-95, wherein the disease or condition is one that adversely affects uterine cell function or viability.

97. The method of claim 96, wherein the disease or condition is endometriosis, uterine fibroids, adenomyosis, a gynecological cancer, pelvic inflammatory disease (PID), cervical dysplasia, or pelvic floor prolapse.

98. The method of any one of claims 86-97, wherein the disease or condition is one that reduces the subject's fertility.

99. The method of claim 98, wherein the disease or condition is embryo implantation failure.

100. The method of any one of claims 86-99, wherein the candidate pharmaceutical intervention is a small molecule, a polynucleotide (e.g., an antisense oligonucleotide, small interfering ribonucleic acid (siRNA), short hairpin RNA (shRNA), or microRNA (miRNA)), a polypeptide (e.g., an antibody or antigen-binding fragment thereof), a gene therapy (e.g., a DNA or RNA vector, optionally wherein the vector is a viral vector), or a cell therapy (e.g., a chimeric antigen receptor T-cell (CAR-T cell) therapy).

101. The method of any one of claims 86-100, wherein the one or more metrics of ovarian and/or uterine function comprise:
a) the rate and/or extent to which the organoid matures an immature oocyte upon co-culturing the organoid with the immature oocyte;
b) the rate and/or extent to which the organoid secretes an estrogen or progestogen, optionally wherein the estrogen is estradiol and/or the progestogen is progesterone;
c) the viability of the organoid or a component cell type thereof; and/or
d) the rate and/or extent to which the organoid specifically binds to one or more cells that model an embryo.

102. The method of claim 101, wherein the immature oocyte is a germinal vesicle (GV)-stage oocyte.

103. The method of claim 101, wherein the immature oocyte is a metaphase I (MI)-stage oocyte.

104. The method of any one of claims 101-103, wherein maturation of the immature oocyte is assessed by evaluating conversion of the immature oocyte into a metaphase II (MII)-stage oocyte.

105. The method of any one of claims 86-104, wherein the one or more metrics of severity of the disease or condition comprise:
a) abnormal or aberrant growth of the organoid or a component cell type thereof;
b) the extent to which the organoid secretes an estrogen or progestogen above or below a reference level of estrogen or progestogen secretion; and/or
c) the rate at which the organoid or a component cell type thereof undergoes necrosis, apoptosis, or other form of cell death.

106. The method of claim 105, wherein the reference level of estrogen or progestogen secretion is a level of estrogen or progestogen secretion observed in a healthy, pre-menopausal human female subject that does not have the disease or condition.

107. The method of claim 105 or 106, wherein the estrogen is estradiol.

108. The method of claim 107, wherein the reference level of estradiol secretion is a concentration of from about 20 pg/ml to about 50 pg/ml.

109. The method of any one of claims 105-108, wherein the progestogen is progesterone.

110. A method of determining whether a candidate pharmaceutical intervention is safe for administration to a female subject having a disease or condition, the method comprising:
a) contacting the candidate pharmaceutical intervention with the organoid of any one of claims 1-68;
b) determining that the organoid does not exhibit (i) a decrease in one or more metrics of ovarian and/or uterine function relative to a measurement of the one or more metrics of ovarian and/or uterine function obtained prior to the contacting, and/or (ii) an increase in one or more indicators of a gynecological disorder relative to a measurement of the one or more metrics of a gynecological disorder obtained prior to the contacting; and, optionally
c) releasing the candidate pharmaceutical intervention for administration to the subject.

111. The method of claim 110, further comprising:
d) administering a therapeutically effective amount of the candidate pharmaceutical intervention to the subject to treat the disease or condition.

112. A method of treating or preventing a disease or condition in a subject (e.g., a female subject) in need thereof, the method comprising:
a) contacting the candidate pharmaceutical intervention with the organoid of any one of claims 1-68;
b) determining that the organoid does not exhibit (i) a decrease in one or more metrics of ovarian and/or uterine function relative to a measurement of the one or more metrics of ovarian and/or uterine function obtained prior to the contacting, and/or (ii) an increase in one or more indicators of a gynecological disorder relative to a measurement of the one or more metrics of a gynecological disorder obtained prior to the contacting; and
c) administering a therapeutically effective amount of the candidate pharmaceutical intervention to the subject.

113. A method of treating or preventing a disease or condition in a subject (e.g., a female subject) in need thereof, the method comprising administering to the subject a therapeutically effective amount of a candidate pharmaceutical intervention, wherein the candidate pharmaceutical intervention has previously been identified as being safe for administration to the subject by a method comprising:
a) contacting the candidate pharmaceutical intervention with the organoid of any one of claims 1-68; and
b) determining that the organoid does not exhibit (i) a decrease in one or more metrics of ovarian and/or uterine function relative to a measurement of the one or more metrics of ovarian and/or uterine function obtained prior to the contacting, and/or (ii) an increase in one or more indicators of a gynecological disorder relative to a measurement of the one or more metrics of a gynecological disorder obtained prior to the contacting.

114. The method of any one of claims 110-113, wherein the subject is a human.

115. The method of any one of claims 110-114, wherein the disease or condition is one that is associated with a decline in ovarian function.

116. The method of any one of claims 110-115, wherein the condition is a timewise reduction in ovarian function.

117. The method of claim 116, wherein the timewise reduction in ovarian function is due to menopause, optionally wherein the menopause is premature menopause.

118. The method of any one of claims 115-117, wherein the decline in ovarian function is a decline in one or more of follicular development, oocyte release, and oocyte maturation.

119. The method of any one of claims 115-118, wherein the disease is POI, PCOS, ovarian cancer, or ovarian hyperstimulation syndrome.

120. The method of any one of claims 110-119, wherein the disease or condition is one that adversely affects uterine cell function or viability.

121. The method of claim 120, wherein the disease or condition is endometriosis, uterine fibroids, adenomyosis, a gynecological cancer, PID, cervical dysplasia, or pelvic floor prolapse.

122. The method of any one of claims 110-121, wherein the disease or condition is one that reduces the subject's fertility.

123. The method of claim 122, wherein the disease or condition is embryo implantation failure.

124. The method of any one of claims 110-123, wherein the candidate pharmaceutical intervention is a small molecule, a polynucleotide (e.g., an antisense oligonucleotide, siRNA, shRNA, or miRNA), a polypeptide (e.g., an antibody or antigen-binding fragment thereof), a gene therapy (e.g., a DNA or RNA vector, optionally wherein the vector is a viral vector), or a cell therapy (e.g., a chimeric antigen receptor T-cell (CAR-T cell) therapy).

125. The method of any one of claims 110-124, wherein the one or more metrics of ovarian and/or uterine function comprise:
a) the rate and/or extent to which the organoid matures an immature oocyte upon co-culturing the organoid with the immature oocyte;
b) the rate and/or extent to which the organoid secretes an estrogen or progestogen, optionally wherein the estrogen is estradiol and/or the progestogen is progesterone;
c) the viability of the organoid or a component cell type thereof; and/or
d) the rate and/or extent to which the organoid specifically binds to one or more cells that model an embryo.

126. The method of claim 125, wherein the immature oocyte is a germinal vesicle (GV)-stage oocyte.

127. The method of claim 125, wherein the immature oocyte is a metaphase I (MI)-stage oocyte.

128. The method of any one of claims 125-127, wherein maturation of the immature oocyte is assessed by evaluating conversion of the immature oocyte into a metaphase II (MII)-stage oocyte.

129. The method of any one of claims 110-128, wherein the one or more indicators of a gynecological disorder comprise:
a) abnormal or aberrant growth of the organoid or a component cell type thereof;
b) the extent to which the organoid secretes an estrogen or progestogen above or below a reference level of estrogen or progestogen secretion; and/or
c) the rate at which the organoid or a component cell type thereof undergoes necrosis, apoptosis, or other form of cell death.

130. The method of claim 129, wherein the reference level of estrogen or progestogen secretion is a level of estrogen or progestogen secretion observed in a healthy, pre-menopausal human female subject that does not have the disease or condition.

131. The method of claim 129 or 130, wherein the estrogen is estradiol.

132. The method of claim 131, wherein the reference level of estradiol secretion is a concentration of from about 20 pg/ml to about 50 pg/ml.

133. The method of any one of claims 129-132, wherein the progestogen is progesterone.

134. A platform for engineering a human organoid replica for reproductive screening, the platform comprising:
a) an engineered reproductive cell;
b) a detector that measures a response by the engineered reproductive cell upon exposure of the engineered reproductive cell to a stimulus;
c) a computing device in communication with the detector, wherein the computing further comprises:
(i) at least a processor; and
(ii) a memory communicatively connected to the processor, the memory containing instructions configuring the at least a processor to process stimulus data received from the detector and compare the stimulus data to a user profile.

135. The platform of claim 134, wherein the engineered reproductive cell is generated utilizing bioprinting.

136. The platform of claim 134, wherein the stimulus relates to drug metabolism.

137. The platform of claim 134, wherein the stimulus relates to a toxicity screen.

138. The platform of claim 134, wherein the stimulus relates to a disease model.

139. The platform of claim 134, wherein the stimulus relates to an epigenetic model.

140. The platform of claim 134, wherein processing the stimulus data comprises utilizing a cell viability assay.

141. The platform of claim 134, wherein processing the stimulus data comprises utilizing a classifier configured to generate an image-based profile assessment.

142. The platform of claim 134, wherein comparing the stimulus data to a user profile comprises generating a reproductive discrepancy treatment plan.

143. The platform of claim 134, further comprising utilizing the engineered human organoid replica for embryo culture peri-implantation modeling.

144. A method for engineering a human organoid replica for reproductive screening, the method comprising:
a) creating a user profile as a function of a reproductive cell relating to a user;
b) receiving a plurality of human iPSCs;
c) recapitulating, in vitro, the reproductive cell utilizing the plurality of human iPSCs; and
d) generating, as a function of the recapitulated reproductive cell, a human organoid replica of an ovary.

145. The method of claim 144, wherein creating the user profile further comprises profiling an ovary at a single cell resolution.

146. The method of claim 145, wherein profiling the ovary further comprises utilizing in silico target discovery.

147. The method of claim 144, wherein creating the user profile further comprises identifying at least a reproductive cell from an ovary, wherein the at least a reproductive cell demonstrates a reproductive discrepancy.

148. The method of claim 147, wherein the reproductive discrepancy comprises a cell health change.

149. The method of claim 147, wherein the reproductive discrepancy comprises endometriosis.

150. The method of claim 144, wherein recapitulating the reproductive cell further comprises replicating a reproductive cell demonstrating a reproductive discrepancy.

151. The method of claim 144, wherein recapitulating the reproductive cell further comprises utilizing transcription factor-directed cell differentiation.

152. The method of claim 144, wherein generating the human organoid replica further comprises generating a human organoid replica containing a reproductive cell demonstrating a reproductive discrepancy.

153. The method of claim 144, wherein generating the human organoid replica further comprises
